(19) United States
(12) Patent Application Publication
(10) Pub. No.: US 2026/0130757 A1
(43) Pub. Date: May 14, 2026

(54) SYSTEMS FOR COVERING METALLIC COMPONENTS IN MEDICAL DEVICES

(71) Applicant: Edwards Lifesciences Corporation, Irvine, CA (US)

(72) Inventors: Olivia Marie Conroy, New Rochelle, NY (US); Shane Chazhikatt Cyriac, Cypress, CA (US); Rani Abdullah Mahmoudi, Huntington Beach, CA (US); David Robert Landon, Huntington Beach, CA (US); Zachary William Gyugyi, Irvine, CA (US); Pablo Hernan Catania, Costa Mesa, CA (US)

(21) Appl. No.: 19/443,028

(22) Filed: Jan. 8, 2026

Related U.S. Application Data
(63) Continuation of application No. PCT/US2024/037181, filed on Jul. 9, 2024.
(60) Provisional application No. 63/525,927, filed on Jul. 10, 2023.

(51) Int. Cl. A61F 2/24

(52) U.S. Cl. CPC A61F 2/2415; A61F 2/2418; A61F 2210/0014; A61F 2220/0033; A61F 2220/0041; A61F 2220/005; A61F 2220/0075; A61F 2240/001; A61F 2250/0037

(57) ABSTRACT

A method for manufacturing an implantable prosthetic device. The method comprises taking a frame having a core and an elongated arm that extends from the core, wherein the arm comprises a tip. The method further comprises taking a compressible cap that includes a shaft having a proximal end and a distal end, an internal cavity extending longitudinally along the shaft from the proximal end toward the distal end, and a head at the distal end. The method further comprises covering at least a portion of the arm with the cap by inserting the tip into the cavity, and, without sewing, affixing the cap to the arm by securing the tip within the cavity.

### CROSS-REFERENCED APPLICATIONS

This application is a Continuation of International Patent Application No. PCT/US 2024/037181 to Conroy et al., filed Jul. 9, 2024, titled “Systems for covering metallic components in medical devices,” which published as WO 2025/014934, and which claims the benefit of U.S. Provisional Application No. 63/525,927, filed on Jul. 10, 2023.

The disclosure of each of the above documents is incorporated by reference in its entirety.

### FIELD OF THE DISCLOSURE

The present disclosed technology generally relates to implantable prosthetic devices having extended appendages, and more specifically to systems and devices for covering a protruding appendage of a prosthetic device.

### BACKGROUND

Cardiovascular prosthetic devices are generally implantable devices for the repair, replacement, and/or assistances of organ systems within the cardiovascular system, including the heart, heart muscle, heart valves, arteries, and veins. A wide variety of cardiovascular conditions are treated with cardiovascular devices, including valvular heart disease, congenital heart defects, arrhythmias, coronary artery disease, and heart failure. Examples of cardiovascular prosthetics include vascular stents, tissue patches, sutures, occluding devices, pacemakers, defibrillators, annuloplasty rings, and prosthetic heart valves.

One emerging method for implanting a cardiovascular prosthetic is a transcatheter technique, which is less invasive and can reduce complications associated with surgical procedures (e.g., open heart surgery). To deliver a prosthetic via a transcatheter technique, the prosthetic can be mounted in a crimped state along the distal end of an elongate delivery catheter and advanced through the vascular system of a patient to the site of implantation. The prosthetic can then be expanded to its functional size at the implantation site, such as by inflating a balloon or utilizing self-expanding stent or frame. Optionally, a prosthetic can have a balloon-expandable, self-expanding, mechanically expandable frame, and/or a frame expandable in multiple or a combination of ways. Prosthetic devices used in this manner include transcatheter heart valves (THV's).

### SUMMARY OF THE DISCLOSURE

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

In some implementations, the techniques described herein relate to a prosthetic, including: a frame including a core and at least one extended arm that extends from the core, such as for anchoring purposes. The extended arm includes a tip and a cap covering the tip, wherein the cap includes a head, a shaft, and cavity extending along a longitudinal axis of the cap. The tip is disposed within the cavity of the cap. The cap may be prefabricated and affixed to the extended arm. The cap may be affixed to the extended arm via an adhesive that bonds the extended arm to an inner surface of the cavity. The cap may be affixed to the extended arm via a fastener that traverses through a side wall of the cap and through a through hole within the extended arm. The cap may be affixed to the extended arm via a band is tightened around the cap.

In some implementations, the cap is affixed to the extended arm by a depositing system. In some implementations, the depositing system may comprise or consist of one or more of: a dipping system, a laminating system, a spraying system, a jet spinning system, and/or an electrospinning system. In some implementations, the cap may be composed of a material with a hardness between about 20 and about 70 of the Shore A hardness scale. In some implementations, the cap may include one or more of the following materials: a polyolefin, a polyamide, polyethylene terephthalate (PET), thermoplastic polyurethane (TPU), expanded polytetrafluoroethylene (ePTFE), siloxanes (e.g., silicone), and acrylics.

In some implementations, the cap includes a cover layer. The cover layer may be formed from thermoplastic polyurethane (TPU).

In some implementations, the head has a greater cross-sectional diameter than the shaft. In certain aspects, a cross-sectional diameter of the head is at least 1.1-times and up to 10-times greater than a cross-sectional diameter of the shaft. In some implementations, the cross-sectional diameter of the head is at least 0.5 mm and up to 10.0 mm. In some implementations, a length of the cap may be between 2.0 mm and 20 mm. In some implementations, a length of a head may be between 0.5 mm and 10 mm. In some implementations, a distance between the distal end of the cavity and the distal end of the cap may be between 0.25 mm and 5.0 mm.

In some implementations, the cap includes a set of fins that extend laterally from the shaft to form the head. In various aspects, the set of fins may include two fins, three fins, four fins, five fins, or six fins. One or more fins may include a bend or a curve in a circumferential direction.

In some implementations, the head includes a set of one or more voids to enhance deformability and compressibility of the head. In some implementations, a set of voids includes a longitudinal notch in a sidewall of the head. In some implementations, the set of voids includes a circumferential notch in a sidewall of the head. In some implementations, the set of voids includes a circular notch in a distal end of the head. In some implementations, the set of voids includes a cavity in a distal end of the head.

In some implementations, the cap is composed of a shape memory polymer wherein the cap includes a first memory state and/or a second memory state. For example, the first memory state may be maintained at a first temperature, wherein the shape memory polymer has a first density, and the cap has a first profile shape. In some implementations, the first temperature may be less than a body temperature. For example, the first temperature may be room temperature, a refrigerated temperature, or a frozen temperature. In some implementations, the first temperature is between about 22 degrees Celsius and 28 degrees Celsius, between about 2 degrees Celsius and 10 degrees Celsius, or below 0 degrees Celsius.

In some implementations, the second memory state is invoked at a second temperature higher than the first temperature. In some implementations, when the cap is within the second memory state, the shape memory polymer preferably has a second density that is less than first density, and the cap has a second profile shape that is a greater profile than the first profile shape. In some implementations, the second temperature is a body temperature of a recipient. In some implementations, the second temperature is between about 35 degrees Celsius and 39 degrees Celsius.

In some implementations, the head has a greater cross-sectional diameter in the second memory state as compared to the first memory state. For example, the head may have a cross-sectional diameter in the second memory state that is at least 1.1-times and up to 10-times greater than a cross-sectional diameter of the head in the first memory state. The head may have a cross-sectional diameter in the second memory state that is at least 2.0 mm and up to 10.0 mm. The head may have a cross-sectional diameter in the first memory state that is at least 0.5 mm and up to 5.0 mm.

In some implementations, the shape memory polymer is polytetrafluoroethylene (PTFE), polylactide (PLA), or ethylene-vinyl acetate (EVA).

In some implementations, the cap includes a stretchable cover layer. The stretchable cover layer may be formed from TPU.

In some implementations, the techniques described herein relate to a prosthetic valve for replacing a native valve of a subject (e.g., a living subject, a simulation, etc.). In some implementations, the prosthetic valve includes a tubular support frame having an inlet end portion and an outlet end portion.

In some implementations, the tubular support frame includes at least one extended arm that extends from a core of the tubular support frame, wherein the at least one extended arm includes a tip. In some implementations, a cap covers the tip. In some implementations, the cap includes a head, a shaft, and/or a cavity extending along a longitudinal axis of the cap. In some implementations, the tip is within the cavity of the cap.

In some implementations, a valve portion positioned within an inner lumen of the tubular support frame. In some implementations, the valve portion includes a plurality of leaflets made from pericardium. In some implementations, the valve portion provides for unidirectional blood through the inner lumen for replacing the function of the native valve.

In some implementations, the prosthetic valve is for replacing a native tricuspid valve or a native mitral valve of the recipient; and wherein the at least one extended arm is an anchor that includes a curve configured for latching onto chordae tendineae upon installation.

In some implementations, the tubular support frame is crimped and contained within a transcatheter system for delivering the prosthetic valve to a site of installation.

In some implementations, the cap is prefabricated and affixed to the extended arm.

In some implementations, the cap is affixed to the extended arm via an adhesive that bonds the extended arm to an inner surface of the cavity.

In some implementations, the cap is affixed to the extended arm via a fastener that traverses through a side wall of the cap and through a through hole within the extended arm.

In some implementations, the cap is affixed to the extended arm via a band is tightened around the cap. The cap may be affixed to the extended arm by a depositing system. In various example, the depositing system may comprise or consist of one or more of a dipping system, a laminating system, a spraying system, a jet spinning system, and/or an electrospinning system.

In some implementations, the cap is composed of a material with a hardness between about 20 and about 70 of the Shore A hardness scale.

In some implementations, the cap includes one or more of the following materials: a polyolefin, a polyamide, polyethylene terephthalate (PET), thermoplastic polyurethane (TPU), expanded polytetrafluoroethylene (ePTFE), siloxanes (e.g., silicone), and acrylics.

In some implementations, the cap includes a cover layer. The cover layer may include TPU.

In some implementations, the head has a greater cross-sectional diameter than the shaft. In some implementations, a cross-sectional diameter of the head may be at least 1.1-times and up to 10-times greater than a cross-sectional diameter of the shaft. In some implementations, a cross-sectional diameter of the head may be at least 0.5 mm and up to 10.0 mm. In some implementations, a length of the cap may be between 2.0 mm and 20 mm. In some implementations, a length of a head may be between 0.5 mm and 10 mm. In some implementations, a distance between the distal end of the cavity and the distal end of the cap may be between 0.25 mm and 5.0 mm.

In some implementations, the cap includes a set of fins that extend laterally from the shaft to yield the head. The set of fins may include two fins, three fins, four fins, five fins, or six fins. In some implementations, one, some, or all of the fins of the set may include a bend or a curve in a circumferential direction.

In some implementations, the head includes a set of one or more voids to enhance deformability and compressibility of the head. In some implementations, the set of voids may include a longitudinal notch in a sidewall of the head. In some implementations, the set of voids may include a circumferential notch in a sidewall of the head. In some implementations, the set of voids may include a circular notch in a distal end of the head. In some implementations, the set of voids may include a cavity in a distal end of the head.

In some implementations, the cap is composed of a shape memory polymer such that the cap includes at least a first memory state. In some implementations, the first memory state may be maintained at a first temperature. In some implementations, when the cap is within the first memory state, the shape memory polymer has a first density, and the cap has a first profile shape. In some implementations, the first temperature may be less than a body temperature of a recipient. In some implementations, the first temperature may be room temperature, a refrigerated temperature, or a frozen temperature. In some implementations, the first temperature may be between about 22 degrees Celsius and 28 degrees Celsius, between about 2 degrees Celsius and 10 degrees Celsius, or below 0 degrees Celsius.

In some implementations, the shape memory polymer of the cap includes a second memory state. The second memory state may be invoked at a second temperature higher than the first temperature. In some implementations, when the cap is within the second memory state, the shape memory polymer has a second density that is less than first density and the cap has a second profile shape that is a greater profile than the first profile shape. In some implementations, the second temperature may be a body temperature of a recipient. In some implementations, the second temperature may be between about 35 degrees Celsius and 39 degrees Celsius.

In some implementations, the head may have a greater cross-sectional diameter in the second memory state as compared to the first memory state. The head may have a cross-sectional diameter in the second memory state that is at least 1.1-times and up to 10-times greater than a cross-sectional diameter of the head in the first memory state. In some implementations, the head may have a cross-sectional diameter in the second memory state that is at least 2.0 mm and up to 10.0 mm. In some implementations, the head may have a cross-sectional diameter in the first memory state that is at least 0.5 mm and up to 5.0 mm. In some implementations, the shape memory polymer may be polytetrafluoroethylene (PTFE), polylactide (PLA), or ethylene-vinyl acetate (EVA). The cap may include a stretchable cover layer. In some implementations, the stretchable cover layer may include TPU.

In some implementations, the techniques described herein relate to a system for fabricating a prosthetic valve for replacing a native valve of a recipient, including a prosthetic valve system including a tubular support frame having an inlet end portion and an outlet end portion. In some implementations, the tubular support frame includes an extended arm (or other elongate member) that extends, such as, from a core of the tubular support frame.

In some implementations, a valve portion may be positioned within an inner lumen of the tubular support frame, wherein the valve portion includes a plurality of leaflets made from pericardium. In some implementations, the valve portion provides unidirectional blood through the inner lumen for replacing the function of the native valve.

In some implementations, a prefabricated cap is configured to cover a tip of the extended arm, wherein the cap includes a head, a shaft, and cavity extending along a longitudinal axis of the cap. In some implementations, the cavity is shaped to receive and engage the extended arm. The cap may serve various purposes, such as for reducing trauma to surrounding tissue. The cap may also provide advantages with respect to ease of manufacturing.

In some implementations, fabrication includes an adhesive for bonding the extended arm to an inner surface of the cavity. In some implementations, fabrication includes a fastener configured to traverse through a side wall of the cap and through a through hole within the extended arm. In some implementations, fabrication includes a band configured to be tightened around the cap.

In some implementations, the prosthetic valve system is for replacing a native tricuspid valve or a native mitral valve of the recipient. The extended arm may be an anchor shaped for capturing or attaching to a native heart valve leaflet during implantation.

In some implementations, the cap is composed of a material with a hardness between about 20 and about 70 of the Shore A hardness scale.

In some implementations, the cap includes one or more of the following materials: a polyolefin, a polyamide, polyethylene terephthalate (PET), thermoplastic polyurethane (TPU), expanded polytetrafluoroethylene (ePTFE), siloxanes (e.g., silicone), and acrylics.

In some implementations, the cap includes a cover layer. For example, the cover layer may include TPU.

In some implementations, the head has a greater cross-sectional diameter than the shaft. a cross-sectional diameter of the head may be at least 1.1-times and up to 10-times greater than a cross-sectional diameter of the shaft. a cross-sectional diameter of the head may be at least 0.5 mm and up to 10.0 mm. A length of the cap may be between 2.0 mm and 20 mm. A length of a head may be between 0.5 mm and 10 mm.

In some implementations, a distance between the distal end of the cavity and the distal end of the cap is between 0.25 mm and 5.0 mm.

In some implementations, the cap includes a set of fins that extend laterally from the shaft to yield the head. In some implementations, the set of fins may include two fins, three fins, four fins, five fins, six fins, or any other suitable number. In some implementations, at least one fin may include a bend or a curve in a circumferential direction.

In some implementations, the head includes a set of one or more voids or cutaway sections to enhance deformability and compressibility of the head. For example, a void may be formed by a longitudinal notch or circumferential notch in a sidewall of the head. In some implementations, a void may also be formed by a circular notch or a cavity along a distal end portion of the head.

In some implementations, the cap is formed at least in part by a shape memory polymer. Using this configuration, the cap may include a first memory state. In some implementations, the first memory state may be maintained at or below a first temperature. In some implementations, when the cap is within the first memory state, the shape memory polymer has a first density and the cap has a first profile shape. In some implementations, the first temperature is less than a body temperature of a recipient. In some implementations, the first temperature may be room temperature, a refrigerated temperature, or a frozen temperature. In some implementations, the first temperature may be between about 22 degrees Celsius and 28 degrees Celsius, between about 2 degrees Celsius and 10 degrees Celsius, or below 0 degrees Celsius.

In some implementations, the shape memory polymer of the cap may include a second memory state. In some implementations, the second memory state may be invoked at a second temperature that is higher than the first temperature. In some implementations, when the cap is in the second memory state, the shape memory polymer has a second density that is less than first density and/or the cap has a second profile shape that has a greater profile than the first profile shape. In some implementations, the second temperature may be a body temperature of a recipient. The second temperature may be between about 35 degrees Celsius and 39 degrees Celsius. In some implementations, the head may have a greater cross-sectional diameter in the second memory state as compared to the first memory state. In some implementations, the head may have a cross-sectional diameter in the second memory state that is at least 1.1-times and up to 10-times greater than a cross-sectional diameter of the head in the first memory state. In some implementations, the head may have a cross-sectional diameter in the second memory state that is at least 2.0 mm and up to 10.0 mm. The head may have a cross-sectional diameter in the first memory state that is at least 0.5 mm and up to 5.0 mm. The shape memory polymer may be polytetrafluoroethylene (PTFE), polylactide (PLA), or ethylene-vinyl acetate (EVA). The cap may include a stretchable cover layer. The stretchable cover layer may include TPU.

In some implementations, the techniques described herein relate to a prefabricated cap for covering a tip of an extended arm of a prosthetic frame. In some implementations, the cap (or sleeve) includes a shaft, and an internal cavity for receiving extended arm or similar component of the frame. In some implementations, the cap or sleeve may include an enlarged distal head portion. In some implementations, the cap may be composed of a material with a hardness between about 20 and about 70 of the Shore A hardness scale.

In some implementations, the cap may include one or more of the following materials: a polyolefin, a polyamide, polyethylene terephthalate (PET), thermoplastic polyurethane (TPU), expanded polytetrafluoroethylene (ePTFE), siloxanes (e.g., silicone), and acrylics. In some implementations, the cap may include a cover layer. For example, the cover layer may include TPU.

In some implementations, the enlarged head portion has a greater cross-sectional diameter than the shaft. In some implementations, a cross-sectional diameter of the head may be at least 1.1-times and up to 10-times greater than a cross-sectional diameter of the shaft.

In some implementations, the techniques described herein relate to a prefabricated cap, wherein a cross-sectional diameter of the head is at least 0.5 mm and up to 10.0 mm. A length of the cap may be between 2.0 mm and 20 mm. A length of a head may be between 0.5 mm and 10 mm. A distance between the distal end of the cavity and the distal end of the cap may be between 0.25 mm and 5.0 mm.

In some implementations, the cap may include a set of fins that extend laterally from the shaft to yield the head. The set of fins may include two fins, three fins, four fins, five fins, or six fins. In some implementations, one, some, or all of the fins of the set may include a bend or a curve in a circumferential direction.

In some implementations, the head may include a set of one or more voids to enhance deformability and compressibility of the head. In some implementations, the set of voids may include a longitudinal notch in a sidewall of the head. In some implementations, the set of voids may include a circumferential notch in a sidewall of the head. The set of voids may include a circular notch in a distal end of the head. The set of voids may include a cavity in a distal end of the head.

In some implementations, the cap is composed of a shape memory polymer such that the cap includes a first memory state. In some implementations, the first memory state may be maintained at a first temperature. In some implementations, when the cap is within the first memory state, the shape memory polymer has a first density, and the cap has a first profile shape. In some implementations, the first temperature may be less than a body temperature of a recipient. In some implementations, the first temperature may be room temperature, a refrigerated temperature, or a frozen temperature. In some implementations, the first temperature may be between about 22 degrees Celsius and 28 degrees Celsius, between about 2 degrees Celsius and 10 degrees Celsius, or below 0 degrees Celsius.

In some implementations, the shape memory polymer of the cap includes a second memory state. In some implementations, the second memory state may be invoked at a second temperature higher than the first temperature. In some implementations, when the cap is within the second memory state, the shape memory polymer has a second density that is less than first density and the cap has a second profile shape that is a greater profile than the first profile shape. In some implementations, the second temperature may be a body temperature of a recipient. In some implementations, the second temperature may be between about 35 degrees Celsius and 39 degrees Celsius.

In some implementations, the head may have a greater cross-sectional diameter in the second memory state as compared to the first memory state. In some implementations, the head may have a cross-sectional diameter in the second memory state that is at least 1.1-times and up to 10-times greater than a cross-sectional diameter of the head in the first memory state. In some implementations, the head may have a cross-sectional diameter in the second memory state that is at least 2.0 mm and up to 10.0 mm. In some implementations, the head may have a cross-sectional diameter in the first memory state that is at least 0.5 mm and up to 5.0 mm. In some implementations, the shape memory polymer may be polytetrafluoroethylene (PTFE), polylactide (PLA), or ethylene-vinyl acetate (EVA). In some implementations, the cap may include a stretchable cover layer. In some implementations, the stretchable cover layer may include TPU.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

### BRIEF DESCRIPTION OF THE DRAWINGS

The description will be more fully understood with reference to the following figures, which are presented as various examples of the disclosure and should not be construed as a complete recitation of the scope of the disclosure.

FIG. 1 provides a schematic of a heart.

FIG. 2 provides an example of a prosthetic frame.

FIGS. 3A and 3B provide an example of cap or cover. FIG. 3A provides a perspective view. FIG. 3B provides a longitudinal cross-sectional view.

FIGS. 4A, 4B-1, 4B-2, and 4B-3 provide an example of a cap with straight fins.

FIG. 5 provides an example of a cap with curved fins.

FIG. 6 provides an example of a cap with wide longitudinal notches.

FIG. 7 provides an example of a cap with narrow longitudinal notches.

FIGS. 8A and 8B provide an example of a cap with circumferential notches. FIG. 8A provides a perspective view. FIG. 8B provides a frontal view.

FIGS. 9A and 9B provide an example of a cap with a circular notch. FIG. 9A provides a perspective view. FIG. 9B provides a longitudinal cross-sectional view.

FIGS. 10A and 10B provide an example of a cap with distal cavities. FIG. 10A provides a perspective view. FIG. 10B provides a longitudinal cross-sectional view.

FIG. 11 provides a schematic of thermosetting a memory shape polymer.

FIGS. 12A and 12B provide an example of a cap composed of a memory shape polymer. FIG. 12A provides a longitudinal cross-sectional view of the cap in a condensed polymer, smaller profile memory state. FIG. 12B provides a longitudinal cross-sectional view of the cap in an expanded polymer, larger profile memory state.

FIG. 13A is a front perspective view of a cap.

FIG. 13B is rear perspective view of the cap.

FIG. 13C is a top-down view of the cap.

FIG. 14A is a top-down view of a cap.

FIG. 14B is a cross sectional view of the cap when installed on a frame of an artificial heart valve.

FIG. 15A is an example cap including micro-barbs.

FIG. 15B is the cap illustrated in FIG. 15A including a shield on top of the micro-barbs.

### DETAILED DESCRIPTION

Medical prosthetic devices may have extended appendages, such as for anchoring. In examples, systems and devices that improve prosthetic manufacturing while reducing stress on the host at the site of installation are described. The systems and devices may be utilized anywhere within a human body, such as within the cardiovascular system. Prosthetic devices may include replacement heart valves, endovascular devices, stents, annuloplasty rings, and docks shaped for receiving another prosthetic device.

In some implementations, a prosthetic system or device includes a set of one or more elongate struts that extend from a prosthetic core or main body. Caps in the form of sleeves or other protective covers are provided over the elongate struts. In some implementations, each cap is configured to cover at least the tip of an extending strut, which can prevent the tip from being exposed. In some implementations, each cap is preferably deformable or otherwise compressible, which can be useful to improve manufacturing efficiency, reduce the profile of the prosthetic device during delivery and/or reduce trauma to surrounding tissue at the implantation site. Implantable prosthetic devices are typically made from biocompatible metals such as nitinol or stainless steel. These metals are well-suited for implantation in the body; however, they can damage surrounding tissue. Especially, when the prosthetic devices have elongated members with protruding tips. As such, it is advantageous to protect the surrounding tissue by providing an atraumatic cap over elongated members.

The described systems, devices and methods should not be construed as limiting in any way. Instead, the present disclosure is directed toward all novel and nonobvious features and aspects of the various disclosed components, alone and in various combinations and sub-combinations with one another. The disclosed systems, devices and methods are not limited to any specific aspect, feature, or combination thereof, nor do the disclosed systems, devices and methods require that any one or more specific advantages be present, or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, devices and methods can be used in conjunction with other systems, devices and methods.

The terms “proximal” and “distal” as used throughout the description relate to a catheter system axis, in which the end where the procedure is performed is the distal end and the opposite end where the catheter system is controlled is the proximal end. Accordingly, the distal end of the catheter system is the leading end that first traverses into the body and first reaches the procedure site. Conversely, the proximal end of the catheter system is the portion that remains extracorporeal. Likewise, a distal movement along the catheter axis would be movement of a component in a direction towards a site of procedure and a proximal movement along the catheter axis would be movement of a component in an opposite direction. Although these terms have a relationship with a site of procedure, it is to be understood that these terms are used for reference and the site of procedure does not need to be present when interpreting the components or movements of the devices and systems described herein.

Various systems and devices for replacement and/or repair are utilized for the purpose of performing a procedure within a recipient. Recipients include (but are not limited to) patients, animal models, cadavers, or anthropomorphic phantoms. Accordingly, in addition to methods of treating patients, the systems and devices can be utilized in training or other practice procedures upon animal models, cadavers, or anthropomorphic phantoms.

The techniques, methods, processes, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.). When tissue or other animal component is utilized, the systems or devices can be further treated with a formaldehyde bioburden reduction process. After preparation, the systems and devices can be stored within a container, which can be hermetically sealed or otherwise kept sterile.

### Overview of Heart and Circulatory Anatomy

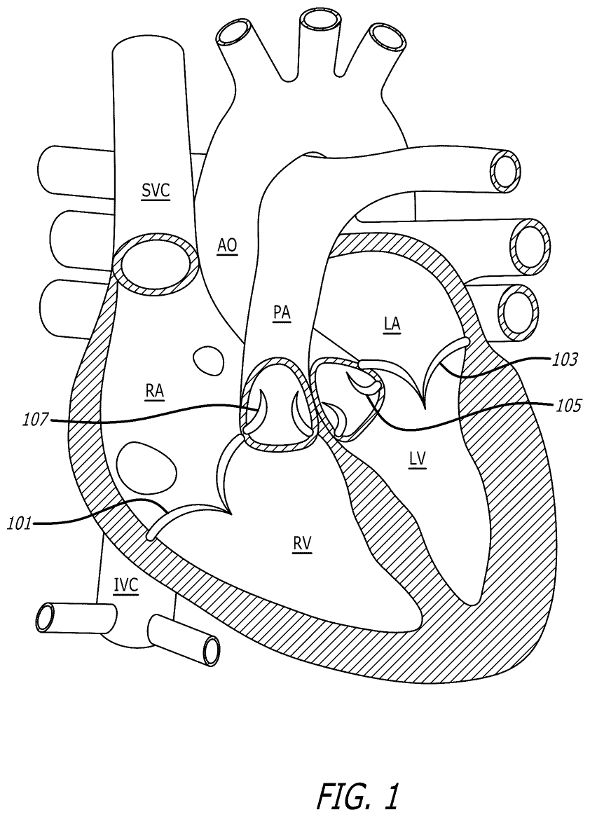

FIG. 1 is a cutaway view of the human heart in a systolic phase. The right ventricle (RV) and left ventricle (LV) are separated from the right atrium (RA) and left atrium (LA), respectively, by the tricuspid valve 101 and mitral valve 103; i.e., the atrioventricular valves. Additionally, the aortic valve 105 separates the LV from the ascending aorta (AO) and the pulmonary valve 107 separates the RV from the pulmonary artery (PA). Each of these valves has flexible leaflets extending inward across the respective orifices that come together or “coapt” in the flowstream to form the one-way, fluid-occluding surfaces.

The RA receives deoxygenated blood from the venous system through the SVC and the IVC, the former entering the RA from above, and the latter from below. During the diastolic phase, or diastole, the deoxygenated blood from the IVC, and SVC that has collected in the RA passes through the tricuspid valve 101 and into the RV as the RV expands. Likewise, oxygenated blood from pulmonary veins that has collected in the LA passes through the mitral valve 103 and into the LV as the LV expands. In the systolic phase, or systole, the RV contracts to force the deoxygenated blood collected in the RV through the pulmonary valve 107 into the pulmonary artery and lungs. Likewise, the LV contracts to force the deoxygenated blood collected in the LV through aortic valve 105 into the aorta and to the peripheral cardiovascular system.

The systems and device described within the present application are described, for illustration, may be utilized within for replacement or repair of any native valve or within the cardiac system. A native valve may need replacement or repair if, for example, the valve is stenotic and/or suffer from insufficiency and/or regurgitation. The systems and devices described herein can be used in various areas whether explicitly described herein or not, as treatment for a defective native valve or another cardiovascular disorder.

### Cardiovascular Prosthetic Systems and Devices

Various cardiovascular prosthetic systems and devices can include a core or main body with a set of one or more extended arms having a tip. The core of the prosthetic system or device can be any core structure for use within a prosthetic device, such as a frame. When utilizing a frame, the frame can comprise interconnected struts that form a plurality of expandable cells or a mesh-like structure. In some implementations, the frame forms a sheet. In some implementations, the frame forms a tubular sheet such that a lumen is formed extending along the longitudinal axis of the tubular form. In some implementations, the core is capable of being expanded and/or crimped such that the length and the width of the core can be altered. In some implementations that comprise a tubular core, expansion and/or crimping of the core can alter the longitudinal axis length and the profile width.

A core can comprise a set of one or more extended arms, wherein each extended arm projects away from the core. The extended arm can be straight or can comprise a curve or a bend. The extended arm can have any of a variety of functions applicable to cardiovascular devices. In some implementations, an extended arm provides structural support for otherwise assists the function of the core. In some implementations, an extended arm can be utilized for interacting with the host at the site of installation. For example, an extended arm can be utilized to anchor a prosthetic device at the site of implantation. In some implementations, a set of one or more extended arms comprises a tip at its extreme end. The tip may be pointed, rounded, or any other shape.

In some implementations, a set of one or more of the extended arms can comprise a cap (e.g., a cover or sleeve) shaped to surround the tip of the extended arm. A cap can provide various benefits, including (but not limited to) preventing exposure of the tip of the extended arm. By covering the tip of the extended arm, the tip is less likely to snag on another component of a prosthetic system or device and prevent unwanted damage to surrounding tissue during or after implantation. In some instances, a cap can be less expensive and easier to fabricate compared to prior techniques that involved sewing of a cloth fabric on and around extended arms. In some implementations, the cap is prefabricated material configured to be fit onto the tip of an extended arm. Methods for fabricating a cap include (but are not limited to) injection molding, thermoforming, and three-dimensional printing. In some implementations, the cap may be applied to the tip of the extended arm through an adhesive such as a silicon adhesive. The adhesive may be cured such that the cap is bonded to the extending arm. The adhesive may be conformable to the extending arm to provide a good bond between the extending arm and the cap. The adhesive may have a Shore A hardness rating of 20. The adhesive may not be fully stiff and may have some give. For example, the adhesive may be twisted 5 to 10 degrees and still twist back. In some implementations, the core of the cap may have a metal press fit which may be press fitted onto the extended arm. In some implementations, the core may be sutured onto the frame. In some implementations, the cap may be directly over molded onto the extended arm.

In some implementations, the cap is fabricated directly on to the extended arm, which can be achieved by a depositing system. Depositing systems that are usable include one or more of (but are not limited to) a dipping system, a laminating system, a spraying system (inclusive of ultrasonic spraying system), a jet spinning system, and/or an electrospinning system.

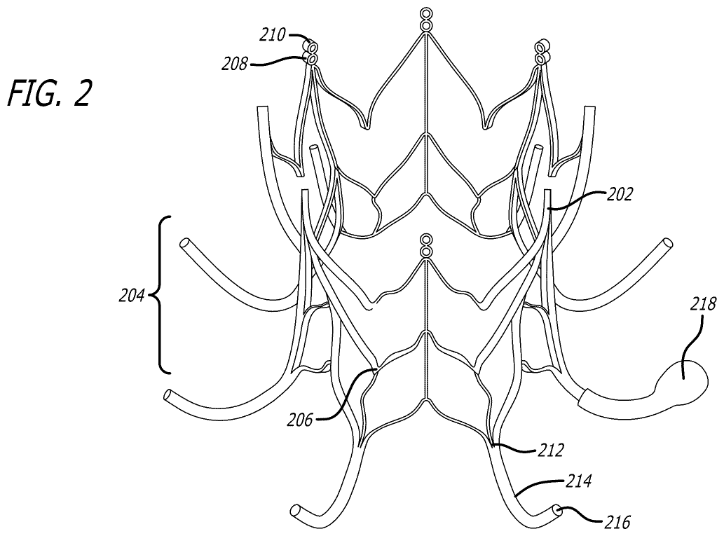

FIG. 2 illustrates an example of a prosthetic tubular frame 202 having a core 204 comprising a set of interconnected struts 206. In the illustrated example, the core 204 provides a substantially cylindrical structure for supporting a one-way valve mechanism. Prosthetic tubular frame 202 comprises a set of extended arms 208 that extend away from core 204 along the longitudinal axis of the tubular frame, each extended arm 208 comprises a tip 210 at the extreme end of the arm. The arms 208 may be used to couple the device to a delivery catheter and/or for attaching a second outer frame. Prosthetic tubular frame 202 also comprises a set of extended anchor arms 212 that extend away from core 204 and include a curve 214, wherein each extended arm 212 comprises a tip 216 at the end of the arm. As illustrated, a cap 218 is provided for covering the tip. In this example, the cap 218 further extends along the extended arm such that it covers a majority of the extended arm, including covering a portion of the curve.

Prosthetic tubular frame 202 is shown to be an expanded configuration. Prosthetic tubular frame 202, however, can be compressed into a crimped configuration, reducing the tubular profile of the frame. The tubular frame can be composed of a biocompatible self-expanding material (e.g., nitinol) or another biocompatible material (e.g., stainless steel) that would require mechanical expansion or a balloon for expansion.

Although prosthetic tubular frame 202 is shown with a particular configuration, any other configuration comprising a core and extended arms can be utilized in various implementations. For instance, the prosthetic frame core can be provided as a sheet that does not form a tube. Further, although extended arm 212 is illustrated at one end of core 204, an extended arm can extend from any portion of the core as long as it extends as an arm with a free point that can be covered. It should be further understood that a cap can be provided to cover a tip of any of the extended arms (208 and 212) of the frame.

Prosthetic tubular frame 202 can be utilized as structural frame for a prosthetic replacement valve for replacing any of the valves of the heart, including the tricuspid, pulmonary, mitral, or aortic valves. A replacement valve can further comprise one or more of an inner skirt, an outer skirt, and a set of leaflets. The tubular frame can have an inlet end portion and outlet end portion with set of leaflets disposed therebetween and within the interior lumen of the prosthetic tubular frame for providing unidirectional blood flow through the valve. The set of leaflets can comprise 2, 3, 4, or more leaflets, which are preferably formed of pericardial tissue derived from bovine, porcine, or human donor. When replacing a tricuspid valve or mitral valve, extended arms 212 can be utilized as an anchor for anchoring the replacement valve. Each extended arm 212 can pass through chordae tendineae of the tricuspid valve or mitral valve for capturing a native leaflet between the arm and tubular frame, thereby resisting atrial and ventricular migration, and helping to hold the replacement valve in place when implanted. It should also be understood that an outer frame (not shown) may surround the tubular frame 202. The outer frame may be used for contacting an annulus. In this case, the native leaflets may be captured in a space between the arms and the outer frame. An outer frame may be attached to the tubular frame via the projections/arms 208 disposed along an upper (e.g., inlet) end of the tubular frame.

Prosthetic tubular frame 202 can be crimped and contained within a sheath of a transcatheter system for delivering the tubular frame. In some implementations, a prosthetic heart valve is crimped and contained within a sheath of a transcatheter system. A prosthetic heart valve can be delivered to a site of installation by any appropriate approach, including (but not limited to) transfemoral, transjugular, subclavian, transapical, or transaortic approach.

### Compressible Caps for Covering Tips

As described herein, a set of one or more of the extended arms can comprise a cap configured to cover the tip of the extended arm, preventing exposure of the tip of the extended arm. In some implementations, a cap for covering the tip of the extended arm is configured to be soft, deformable and/or compressible. The ability to be soft, deformable and/or compressible can have a variety of benefits, including but not limited to reducing stress and damage on host tissue at the site of installation and can yield a smaller profile when the prosthetic is crimped. Reducing profile size can reduce the burden in a transcatheter procedure as the crimped prosthetic can be more easily advanced through the circulatory system via an elongate catheter.

In some implementations, a cap is compressible and/or deformable by utilizing a soft material. In various implementations, the hardness of a cap material can be between about 1 and about 60 of the Shore A hardness scale. In various implementations, as based on the Shore A hardness scale, the hardness of a cap material is between about 1 and about 10, the hardness of a cap material is between about 5 and about 15, the hardness of a cap material is between about 10 and about 20, the hardness of a cap material is between about 15 and about 25, the hardness of a cap material is between about 20 and about 30, the hardness of a cap material is between about 25 and about 35, the hardness of a cap material is between about 30 and about 40, the hardness of a cap material is between about 35 and about 45, the hardness of a cap material is between about 40 and about 50, the hardness of a cap material is between about 45 and about 55, or the hardness of a cap material is between about 50 and about 60.

Various materials can be utilized to provide a soft, deformable and/or compressible cap. For example, in some implementations, the material for a cap is biocompatible and/or bioresorbable. In some implementations, the material for a cap is coated or layered with a material that is biocompatible to form a cover layer. A bioresorbable material may dissolve and be absorbed by the body, such that the component breaks down and degrades over time. Any biocompatible material can be utilized for the various material described herein. Examples of biocompatible and biodegradable material for use as a bioresorbable material include (but are not limited to) poly(glycolic acid) (PGA), poly(lactic acid) (PLA), poly(L-lactic acid) (PLLA), poly(D-lactic acid) (PDLA), poly(D, L-lactic acid) (PDLLA), poly(lactic-co-glycolic acid) (PGLA), poly(β-hydroxybutyrate-co-β-hydroxy valerate) (PHBV), poly(hydroxy butyrate) (PHB), polycaprolactone (PCL), polycyanoacrylates (e.g., poly(octyl cyanoacrylate) (POCA)), polyanhydrides (e. g, poly(fumaric-co-sebacic acid) (p(FASA)), and poly(propylene fumarate) (PPF). The composition of bioresorbable materials by means of various combinations and percentages can be controlled to yield desirable results. For example, degradation time can be controlled via the selection and composition of materials: PGA resorbs within one to two months, PLA/PGA (80/20) resorbs within one to two years, and PLLA resorbs in more than five years.

Materials that can be utilized for a cap include (but are not limited to) polyolefins, polyamides, polyethylene terephthalate (PET), thermoplastic polyurethane (TPU), expanded polytetrafluoroethylene (ePTFE), siloxanes (e.g., silicone), and acrylics. In some instances, polymers are combined and/or blended, which can adjust hardness, deformability, compressibility, and/or resorption rate of a material. For instance, polymers and various siloxanes can be blended or various copolymers can be used. For example, a copolymer of polycarbonate TPU with siloxane, a blend of silica particles with TPU or fluorinated TPUs and PETs can be utilized. In some implementations, a cap includes a cover layer, such as a TPU layer. Any of these substances may be composed of a foam such as a silicone foam.

In some implementations, the cap may be heat shrinkable such that the cap is placed on the extended arm and then heat shrunk to mold onto the extended arm. A heat shrinkable cap has numerous advantages for making manufacturing more efficient and/or providing a secure placement over the arm and/or other strut.

In some implementations, the extended arm may be a molded silicone piece, such as for example, an elongate sleeve. The molded silicone piece may include a wire reinforcement. For instance, the extended arm may include a nitinol wire and an injection molded silicone tube around the nitinol wire. The cap may be an injection molded silicone cap and the tube and cap may be one injection molded piece or two separate pieces. One benefit of making them two separate pieces is that the tube could be a higher durometer than the cap (e.g. higher durometer would make it a little stiffer and better keep the shape of the drop loop; lower durometer on the cap would be softer to the native anatomy and easier to load into the delivery system). The diameter of the silicone tube may be 2 mm or less. The diameter of the cap may be up to 4 mm with cutouts to facilitate easier loading into the delivery system.

Based on the material utilized, the cap and/or cap cover layer can have various advantageous processes, such as (for example) promoting tissue endothelialization and/or mitigating pannus tissue formation. Layers for tissue endothelialization can utilize (for example) polyolefins, polyamides, or PET. Layers for mitigating pannus formation can utilize (for example) TPUs, siloxanes, acrylics, and fluorinated polymers. Various modifications can also promote various properties. For instance, fluorinating polymers (especially TPU) can mitigate pannus formation. Alternatively, coating polymers with amino acids (e.g., lysine or ornithine), with proteins (e.g., collagen, fibronectin, or chitosan), or with growth factors (e.g., VEGF) can promote tissue endothelialization.

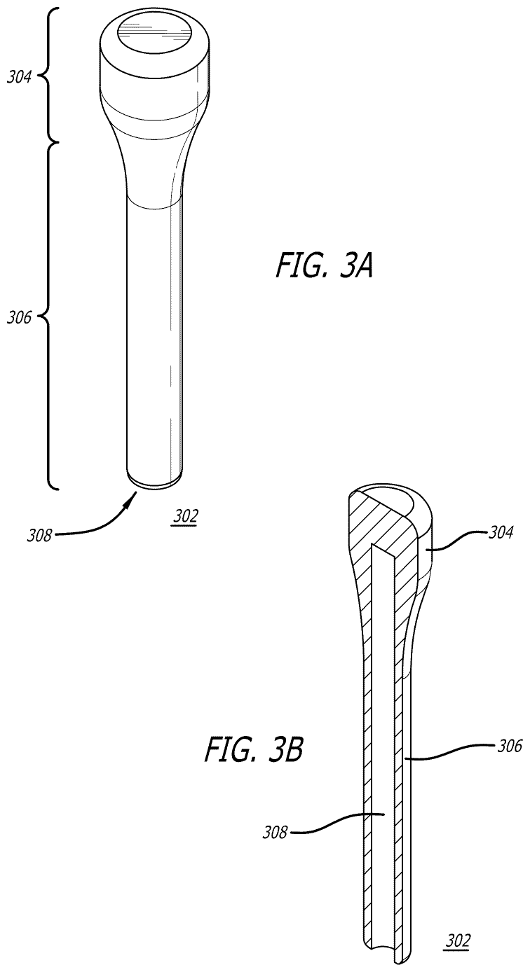

FIGS. 3A and 3B illustrate one example of a cap 302 for covering a tip of an extended arm that extends from prosthetic device. Cap 302 comprises a head 304 at the distal portion of the cap for covering the tip of the extended arm and a shaft 306 at the proximal end of the cap that can cover and extend along the extended arm. When referring to the cap, the distal portion or distal end and the proximal portion and proximal end of the cap is not be confused with the distal-proximal axis of a transcatheter system. Instead, the distal portion or distal end of the cap refers to the portion and end most distal from a prosthetic frame core when installed. And the proximal portion and proximal end of the cap is the portion and end most proximal to a prosthetic frame core when installed.

As shown in this example, the cap can have a cylindrical shape with head 304 having a greater cross-sectional diameter than shaft 306. In some implementations, a cross-sectional diameter of the head is at least 1.1-times and up to 10-times greater than a cross-sectional diameter of the shaft. In various conformations, a cross-sectional diameter of the head is about 1.1-times greater than a cross-sectional diameter of the shaft, a cross-sectional diameter of the head is about 1.5-times greater than a cross-sectional diameter of the shaft, a cross-sectional diameter of the head is about 2.0-times greater than a cross-sectional diameter of the shaft, a cross-sectional diameter of the head is about 3.0-times greater than a cross-sectional diameter of the shaft, a cross-sectional diameter of the head is about 4.0-times greater than a cross-sectional diameter of the shaft, a cross-sectional diameter of the head is about 5.0-times greater than a cross-sectional diameter of the shaft, a cross-sectional diameter of the head is about 10.0-times greater than a cross-sectional diameter of the shaft.

In some implementations, a cross-sectional diameter of a head of a cap is at least 0.5 mm and up to 10.0 mm. In various implementations, a cross-sectional diameter of a head of a cap is between about 0.5 mm and about 1.5 mm, a cross-sectional diameter of a head of a cap is between about 1.0 mm and about 2.0 mm, a cross-sectional diameter of a head of a cap is between about 1.5 mm and about 2.5 mm, a cross-sectional diameter of a head of a cap is between about 2.0 mm and about 3.0 mm, a cross-sectional diameter of a head of a cap is between about 2.5 mm and about 3.5 mm, a cross-sectional diameter of a head of a cap is between about 3.0 mm and about 4.0 mm, a cross-sectional diameter of a head of a cap is between about 3.5 mm and about 4.5 mm, a cross-sectional diameter of a head of a cap is between about 4.0 mm and about 5.0 mm, a cross-sectional diameter of a head of a cap is between about 4.5 mm and about 5.5 mm, a cross-sectional diameter of a head of a cap is between about 5.0 mm and about 6.0 mm, a cross-sectional diameter of a head of a cap is between about 5.5 mm and about 6.5 mm, a cross-sectional diameter of a head of a cap is between about 6.0 mm and about 7.0 mm, a cross-sectional diameter of a head of a cap is between about 6.5 mm and about 7.5 mm, a cross-sectional diameter of a head of a cap is between about 7.0 mm and about 8.0 mm, a cross-sectional diameter of a head of a cap is between about 7.5 mm and about 8.5 mm, a cross-sectional diameter of a head of a cap is between about 8.0 mm and about 9.0 mm, a cross-sectional diameter of a head of a cap is between about 8.5 mm and about 9.5 mm, or a cross-sectional diameter of a head of a cap is between about 9.0 mm and about 10.0 mm.

The contour of the head and/or the shaft can vary such that each can have varying cross-sectional diameters. Furthermore, the cap in various other conformations can have any cross-sectional shape (e.g., quadrilateral, hexagon, etc.). When the cross-sectional shape of a cap is anything other than a circle, references to a cross-sectional diameter as described herein are to apply to the longest diagonal or the longest diameter. And in some implementations, a cap can have a head cross-sectional diameter equal to or less than a shaft cross-sectional diameter.

As depicted, the cap 302 comprises a cavity 308 having an opening at an end opposite of head 304 (i.e., the proximal end of the cap) and extends within and along a longitudinal axis of shaft 306 into the head. Cavity 308 is configured to engage and cover a tip and a portion of an extended arm of a prosthetic frame. Due to the hollow cavity 308, the cap 302 can slide over an extended arm during manufacturing. In some implementations, cavity 308 is configured to provide a snug fit on an extended arm, which can help mitigate retrograde sliding of the cap off the extended arm. In some implementations, the cap is further affixed to an extended arm, which can be achieved by a variety of mechanisms. For example, an adhesive can be applied to bond an extended arm to an inner surface of a cavity. In some implementations, a fastener such as (for example) a suture, a pin, a staple, a rivet, or other fastener can traverse through the cap side wall and a through hole within the extended arm. In some implementations, a band such as (for example) a clamp, ring, suture, wire, brace or other band can be tightened around the cap.

Although a particular configuration for a cap is provided in FIGS. 3A and 3B, other configurations can be utilized in various implementations. For instance, the length of the head, shaft, and/or cavity can be varied in alternative implementations.

In some implementations, the length of a cap is between 2.0 mm and 20 mm. In various implementations, the length of a cap is between about 2.0 mm and about 4.0 mm, the length of a cap is between about 3.0 mm and about 5.0 mm, the length of a cap is between about 4.0 mm and about 6.0 mm, the length of a cap is between about 5.0 mm and about 7.0 mm, the length of a cap is between about 6.0 mm and about 8.0 mm, the length of a cap is between about 7.0 mm and about 9.0 mm, the length of a cap is between about 8.0 mm and about 10.0 mm, the length of a cap is between about 9.0 mm and about 11.0 mm, the length of a cap is between about 2.0 mm and about 4.0 mm, the length of a cap is between about 10.0 mm and about 12.0 mm, the length of a cap is between about 11.0 mm and about 13.0 mm, the length of a cap is between about 12.0 mm and about 14.0 mm, the length of a cap is between about 13.0 mm and about 15.0 mm, the length of a cap is between about 14.0 mm and about 16.0 mm, the length of a cap is between about 15.0 mm and about 17.0 mm, the length of a cap is between about 16.0 mm and about 18.0 mm, the length of a cap is between about 17.0 mm and about 19.0 mm, or the length of a cap is between about 18.0 mm and about 20.0 mm.

In some implementations, the length of a head is between 0.5 mm and 10 mm. In various implementations, the length of a head is between about 0.5 mm and about 1.5 mm, the length of a head is between about 1.0 mm and about 2.0 mm, the length of a head is between about 1.5 mm and about 2.5 mm, the length of a head is between about 2.0 mm and about 3.0 mm, the length of a head is between about 2.5 mm and about 3.5 mm, the length of a head is between about 4.0 mm and about 5.0 mm, the length of a head is between about 4.5 mm and about 5.5 mm, the length of a head is between about 5.0 mm and about 6.0 mm, the length of a head is between about 5.5 mm and about 6.5 mm, the length of a head is between about 6.0 mm and about 7.0 mm, the length of a head is between about 6.5 mm and about 7.5 mm, the length of a head is between about 7.0 mm and about 8.0 mm, the length of a head is between about 7.5 mm and about 8.5 mm, the length of a head is between about 8.0 mm and about 9.0 mm, the length of a head is between about 8.5 mm and about 9.5 mm, or the length of a head is between about 9.0 mm and about 10.0 mm.

In some implementations, the distance between the distal end of the cavity and the distal end of the cap is between 0.25 mm and 5.0 mm. In various implementations, the distance between the distal end of the cavity and the distal end of the cap is between about 0.25 mm and about 0.75 mm, the distance between the distal end of the cavity and the distal end of the cap is between about 0.5 mm and about 1.0 mm, the distance between the distal end of the cavity and the distal end of the cap is between about 0.75 mm and about 1.25 mm, the distance between the distal end of the cavity and the distal end of the cap is between about 1.0 mm and about 1.5 mm, the distance between the distal end of the cavity and the distal end of the cap is between about 1.25 mm and about 1.75 mm, the distance between the distal end of the cavity and the distal end of the cap is between about 1.5 mm and about 2.0 mm, the distance between the distal end of the cavity and the distal end of the cap is between about 1.75 mm and about 2.25 mm, the distance between the distal end of the cavity and the distal end of the cap is between about 2.0 mm and about 2.5 mm, the distance between the distal end of the cavity and the distal end of the cap is between about 2.25 mm and about 2.75 mm, the distance between the distal end of the cavity and the distal end of the cap is between about 2.5 mm and about 3.0 mm, the distance between the distal end of the cavity and the distal end of the cap is between about 2.75 mm and about 3.25 mm, the distance between the distal end of the cavity and the distal end of the cap is between about 3.0 mm and about 3.5 mm, the distance between the distal end of the cavity and the distal end of the cap is between about 3.25 mm and about 3.75 mm, the distance between the distal end of the cavity and the distal end of the cap is between about 3.5 mm and about 4.0 mm, the distance between the distal end of the cavity and the distal end of the cap is between about 3.75 mm and about 4.25 mm, the distance between the distal end of the cavity and the distal end of the cap is between about 4.0 mm and about 4.5 mm the distance between the distal end of the cavity and the distal end of the cap is between about 4.25 mm and about 4.75 mm, or the distance between the distal end of the cavity and the distal end of the cap is between about 4.5 mm and about 5.0 mm.

A head can have various contours and figurations that may be beneficial. In some instances, it is beneficial to have a head with a reduced profile when within a catheter during delivery and an expanded profile when the prosthetic is installed. An expanded profile, especially when the expanded profile is soft, deformable, and compressible, can come in contact with, yet not significantly perturb or injure, the host's local environment at the site of implantation. Accordingly, a number of head implementations can provide an expanded yet soft, deformable or collapsible profile.

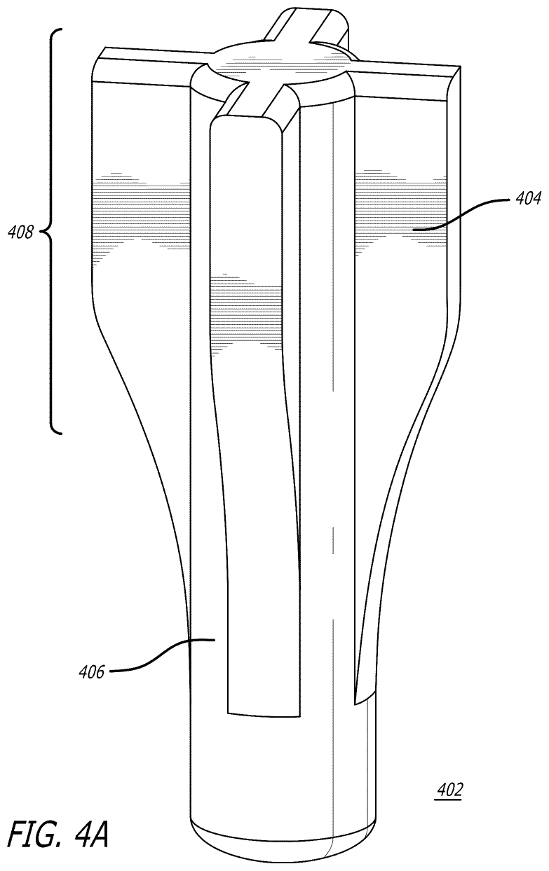

Provided in FIG. 4A is an example of a cap 402 comprising a set of fins 404 that extend laterally from a shaft 406 to yield a head 408. Each fin 404 of the set has a contour such that it laterally extends the furthest one end of cap 402 and tapering down to shaft 406, although other fin contours may be utilized in various implementations. While the example of cap 402 is depicted with four fins, any number of fins can be utilized. In some implementations, a cap has two fins, a cap has three fins, a cap has four fins, a cap has five fins, a cap has six fins, or even more fins. The fin design shown here may provide various benefits. For instance, the fins can be soft and deformable and thus the head can be compressed when a frame is crimped. Further, when installed, the fins can come in contact with the host's local anatomy but not cause significant injury to the local tissue.

In some implementations of cap comprising a set of fins, the fins are provided as straight fins as depicted in the example of FIG. 4A. In other implementations, each fin of the set of fins can include a bend or a curve, such that each fin bends or curves in a circumferential direction. When the set of fins includes a bend or a curve in a circumferential direction, the head compress in a predictable manner when pressure is applied (e.g., when frame valve is crimped within a sheath of a catheter).

Provided in FIGS. 4B-1 and 4B-2 are various views of the cap 402 described in connection with FIG. 4A while implemented on an extended arm 454 of an example prosthetic tubular frame 452. FIG. 4B-1 is a top-down view of the cap 402 while FIG. 4B-2 is a side view of the cap 402. As illustrated, the cap 402 may be oriented such that each of the fins 404 are oriented in an inclined plane from a general plane of extension 454a of the extended arm 454. In some examples, each of the fins 404 may be oriented at a 45-degree angle with respect to the general plane of extension 454a.

Provided in FIG. 4B-3 is a top-down view of the cap 402 described in connection with FIG. 4A while implemented on the example extended arm 454 of the example prosthetic tubular frame 452 when in contact with a surface 456. In some examples, the surface 456 may be a heart tissue when implanted in a patient's body. The surface 456 may be along the septal wall of the patient's heart, specifically the atrium ventricle (e.g. bundle of His). It may be advantageous for any aspect of the cap 402 that comes into contact with the heart tissue to distribute the load over a large surface area to reduce overall pressure and peak pressures. During systole of a patient's heart, the ventricle contracts, thus applying significant load to the contacting portions of the cap 402 (e.g. the fins 404). As illustrated, the fins 404 flatten upon contact with the surface 456, thus distributing the load over a larger area and reducing overall pressure and peak pressures exerted onto the sensitive anatomy of the heart. The cap 404 may be implemented with certain materials which may aid with this flattening. For example, the cap 404 may be a silicone with a certain Shore A hardness or durometer. The Shore A hardness may be hard enough such that the cap 404 does not yield to the prosthetic tubular frame 452 but also provides enough flexibility to flex, as illustrated in FIG. 4B-3 when the fins 404 contact heart tissue. In some implementations, the Shore A hardness of the silicone may be about 20 to about 70. In some implementations, the Shore A hardness may be about 25 to about 60 or about 30 to about 50. In some implementations, the Shore A hardness may be about 30. While the cap 404 is described as silicone, the cap 404 may be made from a material which may be any material which is biocompatible, recognizable with echo, and sterilizable. A material which is not immediately recognizable with echo may be doped to be made recognizable by echo. The cap 404 may be an elastomer. The cap 404 may be an open cell foam.

The cap 404 may include fluoroscopy (fluoro) markers. The flouro markers may be over-molding or embedding into the cap 404. The cap 404 may include radiopaque doped material.

The cap 404 may include a pressure sensor for monitoring pressures. The pressure sensor may be over-molded on the cap 404. The cap 404 may include a pacing electrode which may be utilized to provide pacing to the heart when the artificial heart valve is implanted within the heart.

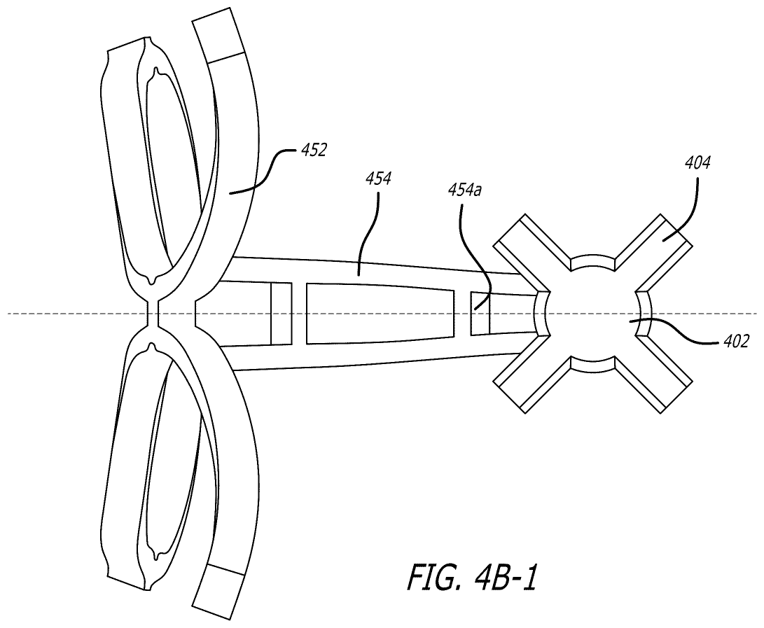

Provided in FIG. 5 is an example of a cap 502 comprising a set of fins 504 that include a curve in a circumferential direction. As depicted, cap 502 includes a set of fins 504 that extend from a shaft 506 to form a head 508. While the example of cap 502 is depicted with six fins, any number of fins can be utilized. In some implementations, a cap has two fins, a cap has three fins, a cap has four fins, a cap has five fins, a cap has six fins, or even more fins.

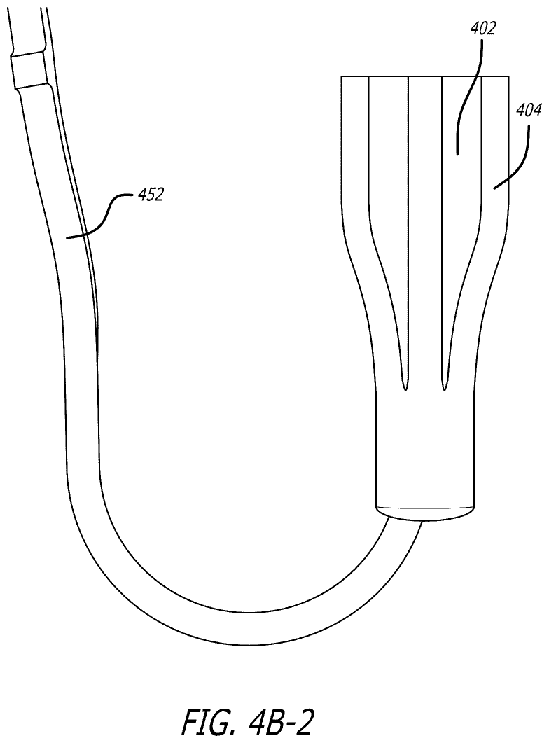

Provided in FIGS. 6 to 10B are various examples of caps that can be utilized in any of a number of implementations. Generally, each cap comprises a head, a shaft, and a cavity. The cavity is configured to engage and cover the tip of and a portion of an extended arm. Each example has a different design for a head, but each design provides void to enhance the deformability and compressibility of the head.

Provided in FIG. 6 is an example of a cap 602 comprising a shaft 604 with a cavity (not shown) and a head 606 having two wide longitudinal notches 608 in the side wall of head that are on opposite sides of the head. Each notch 608 has a depth, a width, and extends a length of head 606, providing void to allow the head to deform and compress.

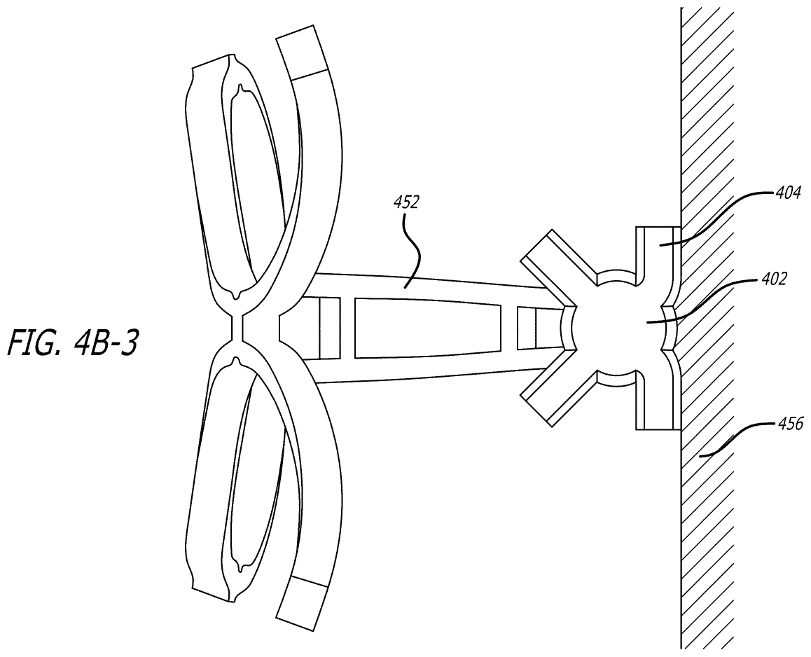

Provided in FIG. 7 is an example of a cap 702 comprising a shaft 704 with a cavity (not shown) and a head 706 having four narrow longitudinal notches 708 in the side wall of the head that are equally spaced along the circumference of the head. Each notch 708 has a depth, a width, and extends a length of head 706, providing void to allow the head to deform and compress.

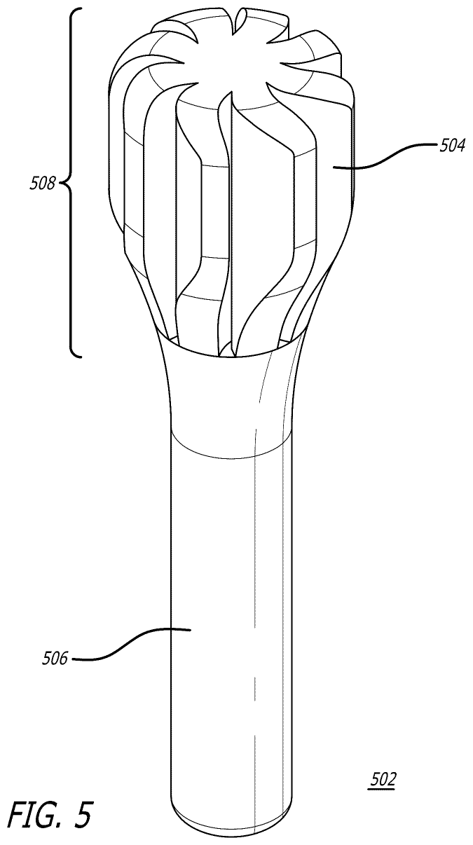

Provided in FIGS. 8A and 8B is an example of a cap 802 comprising a shaft 804 with a cavity (not shown) and a head 806 having three circumferential notches 808 in the side wall of the head that are spaced along the length of the head. Each notch 808 has a depth, a width, and extends along the circumference of head 806, providing void to allow the head to deform and compress.

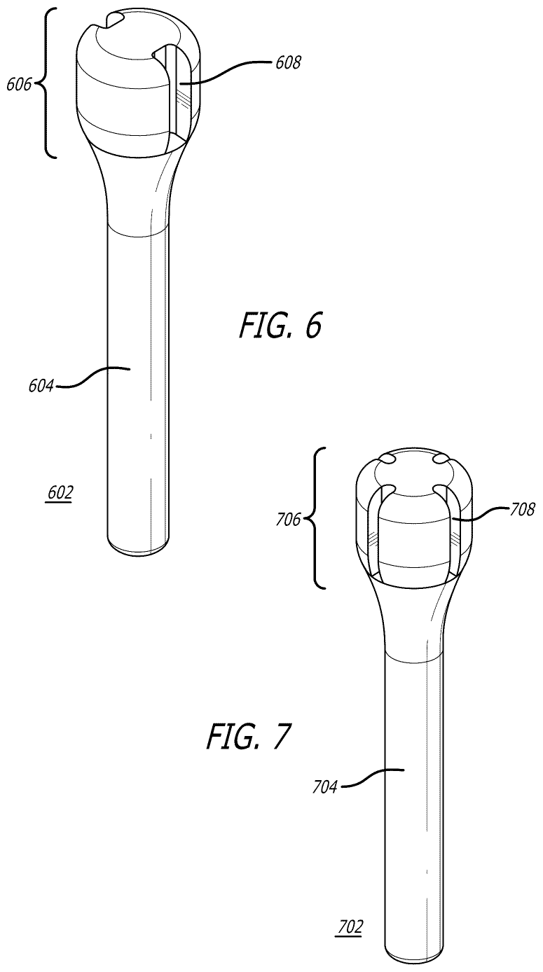

Provided in FIGS. 9A and 9B is an example of a cap 902 comprising a shaft 904 with a cavity 905 and a head 906 having a circular notch 908 in the distal end of the head that is concentric with a circumference of the head. Each notch 908 has a depth, a width, and extends along concentric circle of head 906, providing void to allow the head to deform and compress.

Although the examples in FIGS. 6 to 9B depict notches having particular dimensions, generally, a notch can have any depth, width, and length that provides void that allows the head to deform and compress. Further, although each of the examples have a specific number of notches, a head can have any number of notches that can fit. Accordingly, in various implementations, a head has one notch, a head has two notches, a head has three notches, a head has four notches, a head has five notches, a head has six notches, a head has seven notches, a head has eight notches, a head has nine notches, a head has ten notches, or more notches.

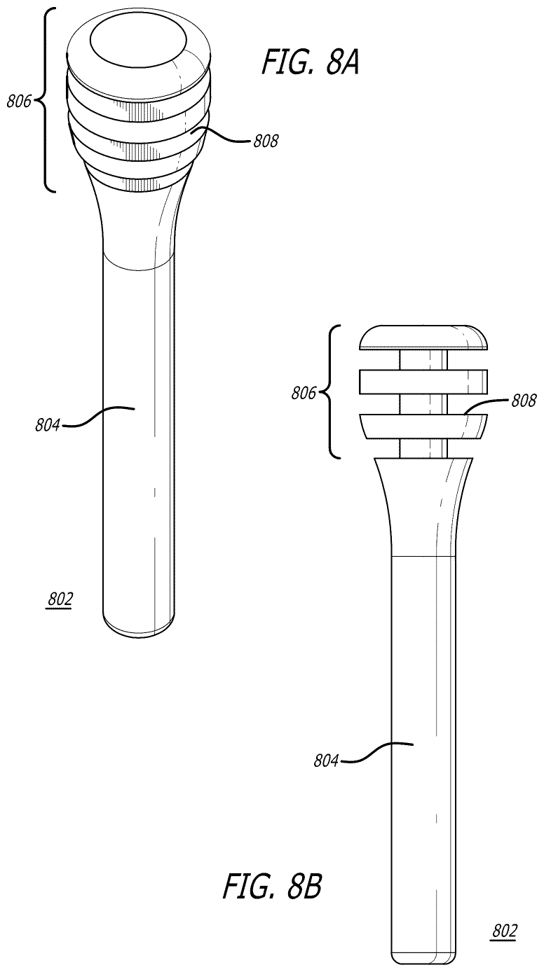

Provided in FIGS. 10A and 10B is an example of a cap 1002 comprising a shaft 1004 with a cavity 1005 and a head 1006 having four cylindrical cavities 1008 in the distal end of the head. Each cavity 1008 has a circumference and extends along a length of head 1006, providing void to allow the head to deform and compress.

Although the example in FIGS. 10A and 10B depict cavities having particular shape and dimensions, generally, a cavity can have any shape and dimensions that provide void that allows the head to deform and compress. Further, although the example has a specific number of cavities, a head can have any number of cavities that can fit. Accordingly, in various implementations, a head has one cavity, a head has two cavities, a head has three cavities, a head has four cavities, a head has five notch cavities, a head has six cavities, a head has seven cavities, a head has eight cavities, a head has nine cavities, a head has ten cavities, or more cavities.

### Caps Composed of Shape Memory Material

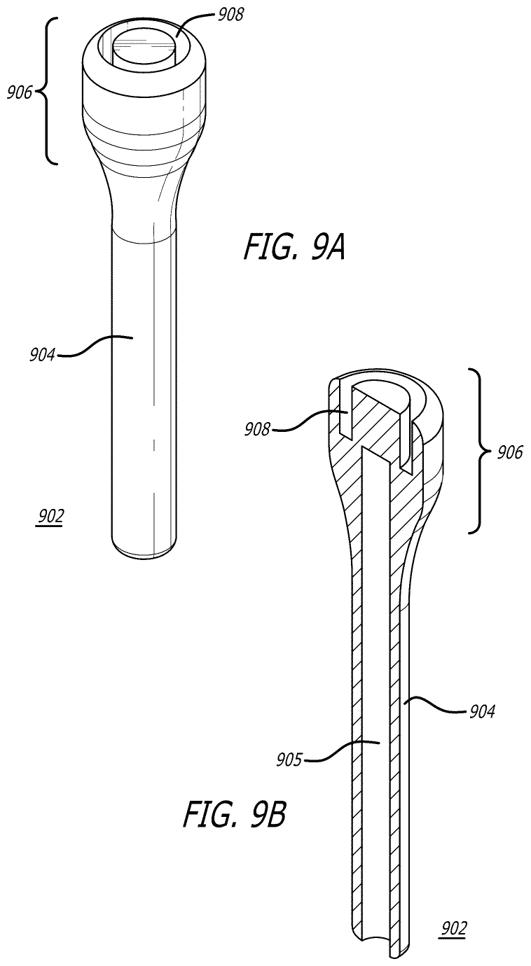

As described herein, a set of one or more of the extended arms can comprise a cap configured to cover the tip of the extended arm, preventing exposure of the tip of the extended arm. In some implementations, a cap for covering the tip of the extended arm is composed of a shape memory material, such as a shape memory alloy (e.g., nitinol) or a shape memory polymer. Shape memory materials may retain two or more shapes and the transition between those shapes is induced by temperature change (FIG. 11).

When using a shape memory alloy, the cap may be formed of a mesh material that expands to an enlarged preset diameter upon activation through placement in the body. The shape memory alloy may be covered with a stretchable outer layer, such as a TPU layer, if desired.

In some implementations, a cap may be formed of a shape memory polymer that is capable of changing shape upon implantation. The polymer can have a first shape at room temperature and a second shape at recipient body temperature. Each shape may have a difference in polymer density, such that a first shape at a lower temperature has a more condensed density of polymer and a second shape at higher temperature has a less condensed density of polymer. Shape memory polymers can be thermoset using a protocol of heat, deformation, and cooling (FIG. 11). In some implementations, the shape memory polymer is formulated as shape memory foam.

In some implementations, a cap is formed utilizing a shape memory polymer such that the cap has at least two memory states. A first memory state can be a condensed polymer having a smaller profile at room temperature. A second memory state can be an expanded polymer having a larger profile at recipient body temperature. The condensed polymer and smaller profile state can be useful for reducing the overall profile size of a tubular frame prosthetic within a sheath of a catheter. Upon installation, the expanded polymer and larger profile state provides a larger yet softer capsule that can mitigate harm to the host at the installation site.

In some implementations, the cap may expand and contract due to hydrophilic properties. The first memory state can be a condensed polymer having a smaller profile at a smaller hydraulic state. The second memory state can be a condensed polymer having a smaller profile at a larger hydraulic state. The shape memory polymer may include a hydrogel or hydrogel-like material.

Various different shape memory polymers are suitable for fabricating a cap. In some implementations, the material for a cap is biocompatible and/or biodegradable. In some implementations, the material for a cap is coated or layered with a material that is biocompatible to form a cover layer. Shape memory polymers that can be utilized for a cap include (but are not limited to) polytetrafluoroethylene (PTFE), polylactide (PLA), and ethylene-vinyl acetate (EVA). In some instances, polymers are combined and/or blended, which can adjust the shape memory ability and/or resorption rate of a material. In some implementations, a cap includes a cover layer, such as a TPU layer. The cover layer can be stretchable such that it can accommodate size changes of a cap composed of shape memory polymer.

Based on the material utilized, the cap and/or cap cover layer can have various advantageous processes, such as (for example) promoting tissue endothelialization and/or mitigating pannus tissue formation. For instance, fluorinating polymers (especially TPU) can mitigate pannus formation. Alternatively, coating polymers with amino acids (e.g., lysine or ornithine), with proteins (e.g., collagen, fibronectin, or chitosan), or with growth factors (e.g., VEGF) can promote tissue endothelialization.

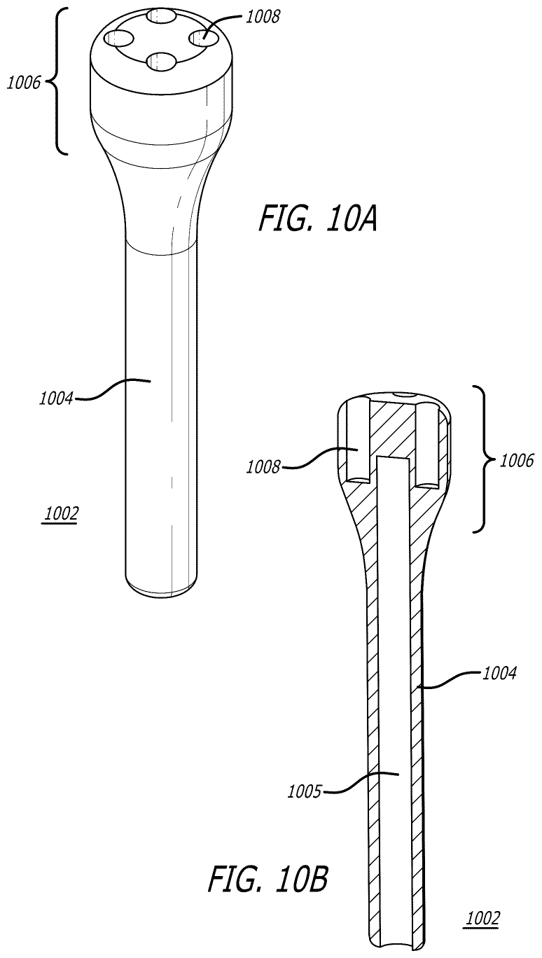

FIGS. 12A and 12B illustrate one example of a cap 1202 for covering a tip of an elongated arm that extends from prosthetic device. In this example, the cap may be formed from a shape memory material, such as nitinol, and/or a shape memory polymer. FIG. 12A depicts cap 1202 at a first memory state at a first temperature, wherein the cap has a smaller shape (e.g., smaller diameter). FIG. 12B depicts cap 1202 at a second memory state at a second temperature, with a larger shape. The first memory state can be maintained a temperature less than a recipient's body temperature (e.g., about 37 degrees Celsius for human recipients). In various implementations, the first memory state is maintained at room temperature, the first memory state is maintained at a refrigerated temperature (i.e., less than room temperature but greater than a frozen temperature), the first memory state is maintained at a frozen temperature. In various implementations, the first memory state is maintained at a temperature between about 22 degrees Celsius and 28 degrees Celsius, the first memory state is maintained at a temperature between about 2 degrees Celsius and 10 degrees Celsius, or the first memory state is maintained at a temperature below 0 degrees Celsius. The second memory state can be invoked by increasing the temperature of the cap to a body temperature of a recipient. In some implementations, the second memory state can be invoked by increasing the temperature of the cap to a temperature between about 35 degrees Celsius and 39 degrees Celsius.

Cap 1202 comprises a head 1204 at the distal portion of the cap for covering the tip of an extended arm and a shaft 1206 at the proximal end of the cap that can cover and extend along the extended arm. When referring to the cap, the distal portion or distal end and the proximal portion and proximal end of the cap is not be confused with the distal-proximal axis of a transcatheter system. Instead, the distal portion or distal end of the cap refers to the portion and end most distal from a prosthetic frame core when installed. And the proximal portion and proximal end of the cap is the portion and end most proximal to a prosthetic frame core when installed.

As shown in this example, the cap can have a cylindrical shape with head 1204 having a greater cross-sectional diameter in the second memory state (FIG. 12B) than the first memory state (FIG. 12A). In some implementations, a cross-sectional diameter of the head in the second memory state is at least 1.1-times and up to 10-times greater than a cross-sectional diameter of the head in the first memory state. In various conformations, a cross-sectional diameter of the head in the second memory state is about 1.1-times greater than a cross-sectional diameter of the head in the second memory state, a cross-sectional diameter of the head in the second memory state is about 1.5-times greater than a cross-sectional diameter of the head in the second memory state, a cross-sectional diameter of the head in the second memory state is about 2.0-times greater than a cross-sectional diameter of the head in the second memory state, a cross-sectional diameter of the head in the second memory state is about 3.0-times greater than a cross-sectional diameter of the head in the second memory state, a cross-sectional diameter of the head in the second memory state is about 4.0-times greater than a cross-sectional diameter of the head in the second memory state, a cross-sectional diameter of the head in the second memory state is about 5.0-times greater than a cross-sectional diameter of the head in the second memory state, a cross-sectional diameter of the head in the second memory state is about 10.0-times greater than a cross-sectional diameter of the head in the second memory state.

In some implementations, a cross-sectional diameter of a head of a cap in the second memory state is at least 2.0 mm and up to 10.0 mm. In various implementations, a cross-sectional diameter of a head of a cap in the second memory state is between about 2.0 mm and about 3.0 mm, a cross-sectional diameter of a head of a cap in the second memory state is between about 2.5 mm and about 3.5 mm, a cross-sectional diameter of a head of a cap in the second memory state is between about 3.0 mm and about 4.0 mm, a cross-sectional diameter of a head of a cap in the second memory state is between about 3.5 mm and about 4.5 mm, a cross-sectional diameter of a head of a cap in the second memory state is between about 4.0 mm and about 5.0 mm, a cross-sectional diameter of a head of a cap in the second memory state is between about 4.5 mm and about 5.5 mm, a cross-sectional diameter of a head of a cap in the second memory state is between about 5.0 mm and about 6.0 mm, a cross-sectional diameter of a head of a cap in the second memory state is between about 5.5 mm and about 6.5 mm, a cross-sectional diameter of a head of a cap in the second memory state is between about 6.0 mm and about 7.0 mm, a cross-sectional diameter of a head of a cap in the second memory state is between about 6.5 mm and about 7.5 mm, a cross-sectional diameter of a head of a cap in the second memory state is between about 7.0 mm and about 8.0 mm, a cross-sectional diameter of a head of a cap in the second memory state is between about 7.5 mm and about 8.5 mm, a cross-sectional diameter of a head of a cap in the second memory state is between about 8.0 mm and about 9.0 mm, a cross-sectional diameter of a head of a cap in the second memory state is between about 8.5 mm and about 9.5 mm, or a cross-sectional diameter of a head of a cap in the second memory state is between about 9.0 mm and about 10.0 mm.

In some implementations, a cross-sectional diameter of a head of a cap in the first memory state is at least 0.5 mm and up to 5.0 mm. In various implementations, a cross-sectional diameter of a head of a cap in the first memory state is between about 0.5 mm and about 1.5 mm, a cross-sectional diameter of a head of a cap in the first memory state is between about 1.0 mm and about 2.0 mm, a cross-sectional diameter of a head of a cap in the first memory state is between about 1.5 mm and about 2.5 mm, a cross-sectional diameter of a head of a cap in the first memory state is between about 2.0 mm and about 3.0 mm, a cross-sectional diameter of a head of a cap in the first memory state is between about 2.5 mm and about 3.5 mm, a cross-sectional diameter of a head of a cap in the first memory state is between about 3.0 mm and about 4.0 mm, a cross-sectional diameter of a head of a cap in the first memory state is between about 3.5 mm and about 4.5 mm, or a cross-sectional diameter of a head of a cap in the first memory state is between about 4.0 mm and about 5.0 mm.

The contour of the head and/or the shaft can vary such that each can have varying cross-sectional diameters. Furthermore, the cap in various other conformations can have any cross-sectional shape (e.g., quadrilateral, hexagon, etc.). When the cross-sectional shape of a cap is anything other than a circle, references to a cross-sectional diameter as described herein are to apply to the longest diagonal or the longest diameter. And in some implementations, a cap can have a head cross-sectional diameter equal to or less than a shaft cross-sectional diameter.

As depicted, cap 1202 comprises a cavity 1208 having an opening at an end opposite of head 1204 (i.e., the proximal end of the cap) and extends within and along a longitudinal axis of shaft 1206 into the head. Cavity 1208 is configured to engage and cover a tip and a portion of an extended arm of a prosthetic frame. Via cavity 1208, cap 1202 can slide onto a tip of and along an extended arm. In some implementations, cavity 1208 is configured to provide a snug fit on an extended arm, which can help mitigate retrograde sliding of the cap off the extended arm. In some implementations, the cap is further affixed to an extended arm, which can be achieved by a variety of mechanisms. For example, an adhesive can be applied to bond an extended arm to an inner surface of a cavity. In some implementations, a fastener such as (for example) a suture, a pin, a staple, a rivet, or other fastener can traverse through the cap side wall and a through hole within the extended arm. In some implementations, a clamp, ring, suture, wire, brace or other band can be tightened around the cap.

Although a particular configuration for a cap is provided in FIGS. 12A and 12B, other configurations can be utilized in various implementations. For instance, the length of the head, shaft, and/or cavity can be varied in alternative implementations to suit a particular purpose.

In some implementations, the length of a cap is between 2.0 mm and 20 mm. In various implementations, the length of a cap is between about 2.0 mm and about 4.0 mm, the length of a cap is between about 3.0 mm and about 5.0 mm, the length of a cap is between about 4.0 mm and about 6.0 mm, the length of a cap is between about 5.0 mm and about 7.0 mm, the length of a cap is between about 6.0 mm and about 8.0 mm, the length of a cap is between about 7.0 mm and about 9.0 mm, the length of a cap is between about 8.0 mm and about 10.0 mm, the length of a cap is between about 9.0 mm and about 11.0 mm, the length of a cap is between about 2.0 mm and about 4.0 mm, the length of a cap is between about 10.0 mm and about 12.0 mm, the length of a cap is between about 11.0 mm and about 13.0 mm, the length of a cap is between about 12.0 mm and about 14.0 mm, the length of a cap is between about 13.0 mm and about 15.0 mm, the length of a cap is between about 14.0 mm and about 16.0 mm, the length of a cap is between about 15.0 mm and about 17.0 mm, the length of a cap is between about 16.0 mm and about 18.0 mm, the length of a cap is between about 17.0 mm and about 19.0 mm, or the length of a cap is between about 18.0 mm and about 20.0 mm.

In some implementations, the length of a head is between 0.5 mm and 10 mm. In various implementations, the length of a head is between about 0.5 mm and about 1.5 mm, the length of a head is between about 1.0 mm and about 2.0 mm, the length of a head is between about 1.5 mm and about 2.5 mm, the length of a head is between about 2.0 mm and about 3.0 mm, the length of a head is between about 2.5 mm and about 3.5 mm, the length of a head is between about 4.0 mm and about 5.0 mm, the length of a head is between about 4.5 mm and about 5.5 mm, the length of a head is between about 5.0 mm and about 6.0 mm, the length of a head is between about 5.5 mm and about 6.5 mm, the length of a head is between about 6.0 mm and about 7.0 mm, the length of a head is between about 6.5 mm and about 7.5 mm, the length of a head is between about 7.0 mm and about 8.0 mm, the length of a head is between about 7.5 mm and about 8.5 mm, the length of a head is between about 8.0 mm and about 9.0 mm, the length of a head is between about 8.5 mm and about 9.5 mm, or the length of a head is between about 9.0 mm and about 10.0 mm.

In some implementations, the distance between the distal end of the cavity and the distal end of the cap is between 0.25 mm and 5.0 mm. In various implementations, the distance between the distal end of the cavity and the distal end of the cap is between about 0.25 mm and about 0.75 mm, the distance between the distal end of the cavity and the distal end of the cap is between about 0.5 mm and about 1.0 mm, the distance between the distal end of the cavity and the distal end of the cap is between about 0.75 mm and about 1.25 mm, the distance between the distal end of the cavity and the distal end of the cap is between about 1.0 mm and about 1.5 mm, the distance between the distal end of the cavity and the distal end of the cap is between about 1.25 mm and about 1.75 mm, the distance between the distal end of the cavity and the distal end of the cap is between about 1.5 mm and about 2.0 mm, the distance between the distal end of the cavity and the distal end of the cap is between about 1.75 mm and about 2.25 mm, the distance between the distal end of the cavity and the distal end of the cap is between about 2.0 mm and about 2.5 mm, the distance between the distal end of the cavity and the distal end of the cap is between about 2.25 mm and about 2.75 mm, the distance between the distal end of the cavity and the distal end of the cap is between about 2.5 mm and about 3.0 mm, the distance between the distal end of the cavity and the distal end of the cap is between about 2.75 mm and about 3.25 mm, the distance between the distal end of the cavity and the distal end of the cap is between about 3.0 mm and about 3.5 mm, the distance between the distal end of the cavity and the distal end of the cap is between about 3.25 mm and about 3.75 mm, the distance between the distal end of the cavity and the distal end of the cap is between about 3.5 mm and about 4.0 mm, the distance between the distal end of the cavity and the distal end of the cap is between about 3.75 mm and about 4.25 mm, the distance between the distal end of the cavity and the distal end of the cap is between about 4.0 mm and about 4.5 mm the distance between the distal end of the cavity and the distal end of the cap is between about 4.25 mm and about 4.75 mm, or the distance between the distal end of the cavity and the distal end of the cap is between about 4.5 mm and about 5.0 mm.

### Caps With a Paddle or T-Shape

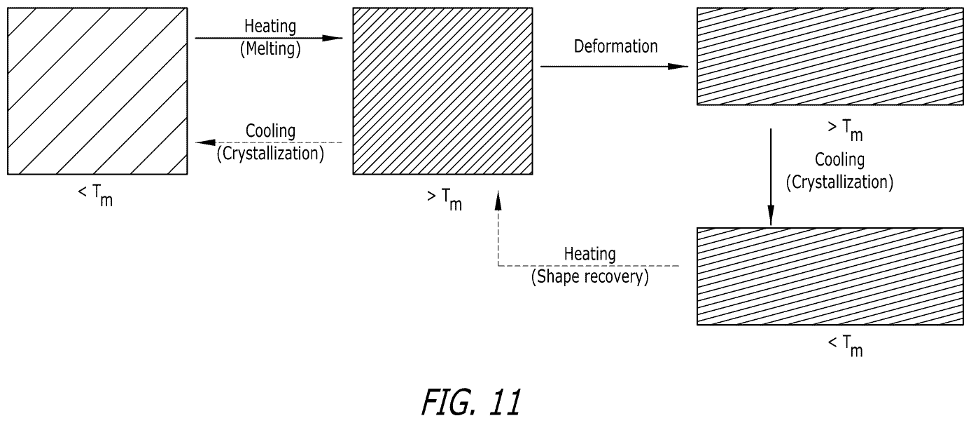

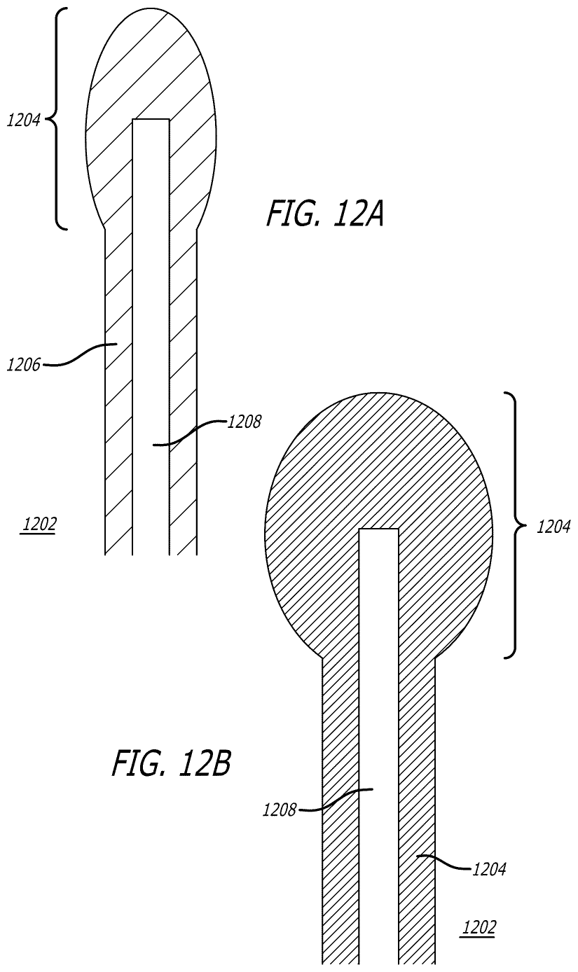

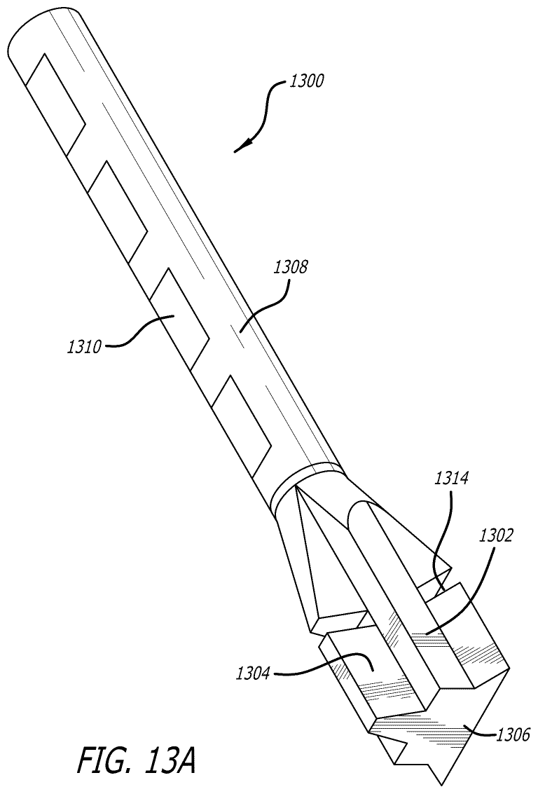

In some implementations, the compressible caps may include fins with a splayed position to form a paddle or T shape. In some examples, the compressible caps may have a pointed side and a flat side. FIG. 13A is a front perspective view of a cap. FIG. 13B is rear perspective view of the cap. FIG. 13C is a top-down view of the cap. The cap 1300 includes a top portion 1302 and a bottom portion 1308. The top portion includes a flat side 1306 and a pointed side 1304. When the tip 1300 is installed on the frame of a cardiovascular prosthetic device and implanted in a heart, the pointed side 1304 may cooperate with the frame (or core) of the artificial heart valve to pinch the native leaflet of the heart between the pointed side 1304 and the frame (or core). In some cases, a prosthetic heart valve includes an inner and an outer frame. In that case, the elongated arms are anchors covered by caps, which trap native leaflets between the outer frame and the caps.

The flat side 1306 of the cap 1300 may touch the ventricle walls and may include a large surface area which may reduce damage to the ventricle walls. The bottom portion 1308 may be extended such that a substantial portion of the tip of the frame is fitted within the bottom portion 1308. The extended bottom portion may include a rectangular shape which matches the shape of the tip of the frame. The bottom portion 1308 may include relief cuts such that when installed on the tip of the frames of the artificial heart valve, the relief cuts 1310 allow the bottom portion 1308 to easily bend onto a curved portion of the frame. The top portion 1302 may include an indentation 1314. The indentation 1314 may be configured to accommodate a suture which holds the cap 1300 on the frame.

It will be understood that a cap having a wide or T-shaped end could have advantages. For example, the forces would be distributed over a larger area, thereby reducing the pressure on any particular region of native tissue. A wider end could also more effectively capture a native leaflet and would be less likely to inadvertently slip through surrounding chordae. In some implementations, the T-shaped ends could be made to extend laterally over time, such as by using a hydrophilic swellable material.

### Elongated Members Forming Caps

In some examples, the caps may be elongated members which wrap around the tips of the frame. The elongated members may include protrusions or tabs. The cap may be a flat material that locks to a surface of an anchor strut. The flat material may not surround or encapsulate the strut but rather just attaches to surface of the anchor strut. The flat material may be a pad that attaches or adheres to the surface. In some cases, the flat material may not wrap around the tip. The elongated member may include two enlarged heads that snap through holes in the anchor for securing the pad to the anchor. However, this is just one way of attaching the elongated member. The elongated member could be sutured or glued in order to hold the elongated member on the anchor.

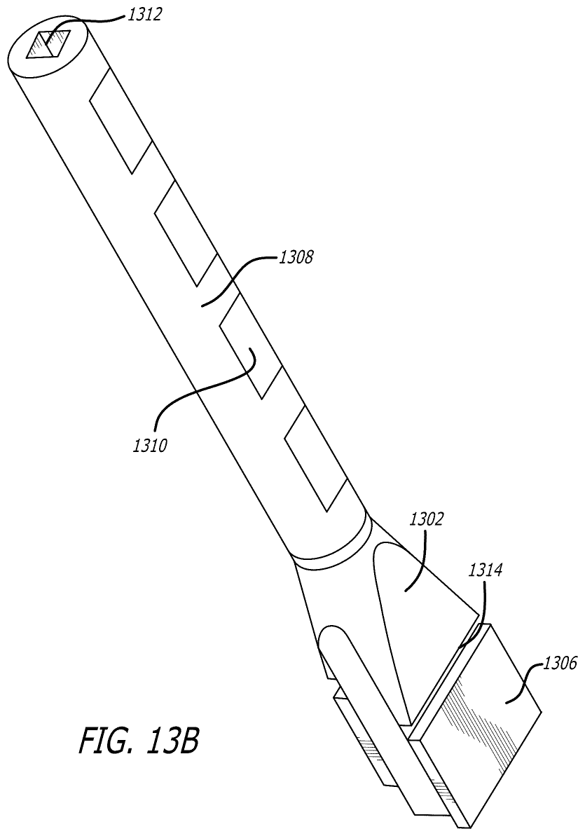

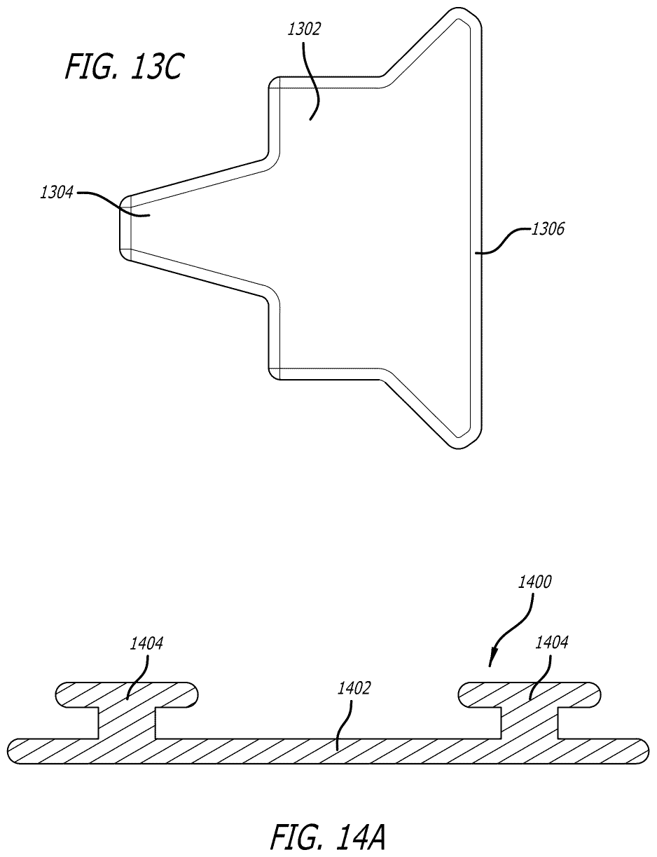

FIG. 14A is a top-down view of a cap. FIG. 14B is a cross-sectional view of the cap when installed on a frame of an artificial heart valve. The cap 1400 includes an elongated member 1402. Each end of the elongated member 1402 includes a protrusion 1404. As illustrated in FIG. 14B, when installed on a frame, the elongated member 1402 wraps over a distal portion of a tip 1406 of the frame such that a middle section of the elongated member 1402 bends over the tip 1406 to cover the sharp end of the tip 1406 of the frame. One of the protrusions 1404 wraps around the tip 1406 of the frame in a direction perpendicular to a major extending direction of the elongated member 1402 and adjacent to the sharp end of the tip 1406. The other of the protrusions 1404 wraps around the frame in a direction perpendicular to the major extending direction of the elongated member 1402 at a location proximal to a curving portion of the tip 1406 of the frame.

### Caps Including Micro-Barbs

In some examples, the caps may include micro-barbs on the surface. These micro-barbs may be over molded on the surface of the caps. The micro-barbs may aid retention of the artificial heart valve when implanted in a heart. The deformable surfaces of the cap may allow the micro-barbs to engage only under designed pressure or load. The micro-barbs may only engage when exposed and shielded when retention is undesirable.

The micro-barbs may be friction elements used for retention to help the caps engage in the native anatomy. These micro-barbs may be thin nitinol wires that poke out of the caps and face the ventricular anatomy after implantation. In some examples, the micro-barbs may be hardened plastics that are shaped to a point or Velcro-like textiles. The micro-barbs may create friction with the native anatomy that may help the cap and thus the prosthetic stay in place. The micro-barbs may be frictional anchoring elements to the surrounding anatomy that only engage the anatomy when sufficient radial force is applied. The micro-barbs may be Velcro-like barbs and made of metal such as NiTi or CoCr or may be a polymer. The polymer micro-barbs may be less adhering.

The micro-barbs may be encapsulated in a shield. The micro-barbs may be over molded into the shield with a compressible void that would allow the outer surface of the shield to deform under pressure and expose the micro-barbs. During implantation of the prosthetic, the micro-barbs do not engage while navigating the anatomy but engage only when they apply radial force to the anatomy. If the micro-barbs are placed and oriented towards the anatomy when deployed, they may not see compression during valve loading as the implantation capsule interacts with the opposing side of the anchor.

As discussed above, the shield may be included on top of the micro-barbs during implantation. The shield may be utilized during valve crimping so that these micro-barbs do not contact a surface of a capsule liner which may cause difficulty deploying the prosthetic. The shield may be extra foam or silicone over those micro-barbs so that whenever the cap is in compression (e.g. during loading), the micro-barbs may be hidden behind that non-sharp foam/silicone surface. Once the valve is deployed and the anchor tips are in their relaxed or tensioned state, these micro-barbs then extend beyond the foam/silicone surface and may interact with the anatomy.

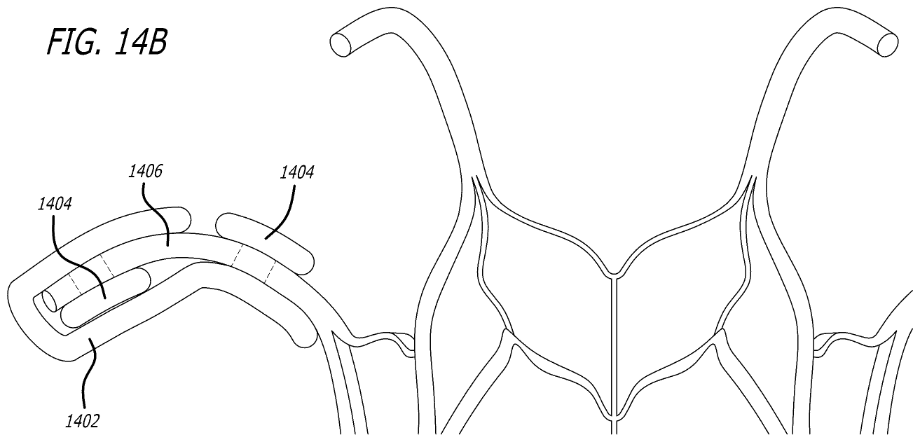

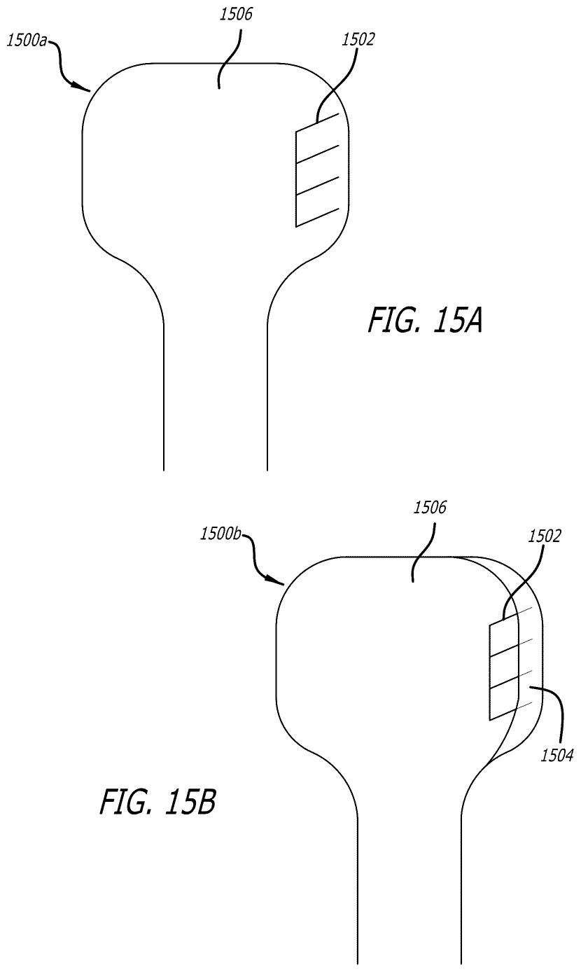

FIG. 15A is an example cap including micro-barbs. The cap 1500a includes micro-barbs 1502 which are coated on the surface of the cap 1500 a. The micro-barbs 1502 may have a main branch that extends vertically and a plurality of sub-branches that extend horizontally from various portions of the main branch. FIG. 15B is the cap 1500a illustrated in FIG. 15A including a shield 1504 on top of the micro-barbs 1502. The shield 1504 disables the effectiveness of the micro-barbs 1502 from adhering to tissue. The shield 1504 may be foam or silicone over the micro-barbs 1502. The shield 1504 may hide the micro-barbs 1502 while the cap is loaded in an encapsulation capsule. The shield 1504 may allow the micro-barbs 1502 to be hidden and thus not engage with anatomy but then engage with anatomy when the micro-barbs apply radial force to the anatomy.

### DOCTRINE OF EQUIVALENTS

While the above description contains many specific implementations of the disclosure, these should not be construed as limitations on the scope of the disclosure, but rather as an example of one implementation thereof. It is therefore to be understood that the present disclosure may be practiced in ways other than specifically described, without departing from the scope and spirit of the present disclosure. Thus, implementations of the present disclosure should be considered in all respects as illustrative and not restrictive. Accordingly, the scope of the disclosure should be determined not by the implementations illustrated, but by the appended claims and their equivalents.

1. A method for manufacturing an implantable prosthetic device, the method comprising:
taking a frame having a core and an elongated arm that extends from the core, wherein the arm comprises a tip;
taking a compressible cap that includes a shaft having a proximal end and a distal end, an internal cavity extending longitudinally along the shaft from the proximal end toward the distal end, and a head at the distal end,
covering at least a portion of the arm with the cap by inserting the tip into the cavity, and
without sewing, affixing the cap to the arm by securing the tip within the cavity.

2. The method according to claim 1, further comprising fabricating the cap prior to the step of covering.

3. The method according to claim 2, wherein fabricating the cap comprises fabricating the head to include at least one void that enhances deformability of the head.

4. The method according to claim 3, wherein the at least one void includes at least one longitudinal notch, and wherein fabricating the head includes fabricating the head to have the at least one longitudinal notch.

5. The method according to claim 3, wherein the at least one void includes at least one circumferential notch, and wherein fabricating the head includes fabricating the head to have the at least one circumferential notch.

6. The method according to claim 2, wherein fabricating the cap comprises forming at least two laterally-extending fins that define the head.

7. The method according to claim 2, further comprising coating the cap with a cover layer.

8. The method according to claim 2, wherein fabricating the cap comprises fabricating the cap from a stretchable material.

9. The method according to claim 2, wherein fabricating the cap comprises fabricating the cap from a shape memory material.

10. The method according to claim 2, wherein fabricating the cap comprises fabricating the cap from a material having a hardness between about 20 and about 70 on the Shore A hardness scale.

11. The method according to claim 2, wherein fabricating the cap comprises fabricating the cap from a material having a hardness between about 1 and about 60 of the Shore A hardness scale.

12. The method according to claim 1, wherein the tip is dimensioned to snugly fit within the cavity, and wherein inserting the tip into the cavity comprises snugly inserting the tip into the cavity.

13. The method according to claim 1, wherein affixing the cap comprises applying adhesive between the arm and an inner surface of the cavity.

14. The method according to claim 1, wherein affixing the cap comprises inserting a fastener to traverse through a side wall of the cap and through a through hole within the arm.

15. The method according to claim 1, wherein affixing the cap comprises tightening a band around the cap.

16. The method according to claim 1, wherein affixing the cap comprises heat-shrinking the cap onto the arm.

17. The method according to claim 1, wherein fabricating the cap comprises fabricating the cap such that the head is wider than the shaft.

18. The method according to claim 1, wherein:
the elongated arm is a first elongated arm,
the frame further has a second elongated arm,
the cap is a first cap,
the head is a first-cap head, and
the method further comprises:
covering at least a portion of the second arm with a second cap that has a second-cap head that is shaped differently to the first-cap head, and
without sewing, affixing the second cap to the second arm.

19. The method according to claim 1, wherein the head includes lateral fins, and wherein affixing the cap to the arm comprises affixing the cap to the arm such that the lateral fins are configured to splay flat responsively to contact with tissue.

20. An implantable prosthetic device manufactured according to the method of claim 1.
